SEQUENCE LISTING (62 sequences)

SEQ ID NO: 1 - protein, 10 aa, synthetic construct
GXISSNNVMG

SEQ ID NO: 2 - protein, 16 aa, synthetic construct
SXTDSDLANYAASVKG

SEQ ID NO: 3 - protein, 10 aa, synthetic construct
TXMTYPPXIS

SEQ ID NO: 4 - protein, 10 aa, synthetic construct
GVSFSRTSLG

SEQ ID NO: 5 - protein, 17 aa, synthetic construct
VISWIGGTTYYADSLKG

SEQ ID NO: 6 - protein, 14 aa, synthetic construct
SNGVLPRTEGAFAS

SEQ ID NO: 7 - protein, 10 aa, synthetic construct
EGTFRTNVMG

SEQ ID NO: 8 - protein, 17 aa, synthetic construct
AISWSGGITHYSNSVKG

SEQ ID NO: 9 - protein, 18 aa, synthetic construct
DPSTTPWNLXXXVNEYEY

SEQ ID NO: 10 - protein, 25 aa, synthetic construct
QVQLVXSGGGLVQPGGSLRLSCAAS

SEQ ID NO: 11 - protein, 14 aa, synthetic construct
WYRQGPGKQREFVA

SEQ ID NO: 12 - protein, 32 aa, synthetic construct
RFTISRDNAKNTVNLQMNSLRPEDTXVYYCRL

SEQ ID NO: 13 - protein, 32 aa, synthetic construct
RFTISRDNAKNTVNLQMNSLRPEDTXVYYCNA

SEQ ID NO: 14 - protein, 11 aa, synthetic construct
WGQGTQVTVSS

SEQ ID NO: 15 - protein, 25 aa, synthetic construct
QVQLVXSGGGLVQAGGSLRLSCAAS

SEQ ID NO: 16 - protein, 14 aa, synthetic construct
WFRQAPGKEREFVS

SEQ ID NO: 17 - protein, 32 aa, synthetic construct
RFTISRDNAKSTVYLQMNSLKPEDTAXYYCAX

SEQ ID NO: 18 - protein, 25 aa, synthetic construct
QVQLVQSGGGLVQAGDSLRLACAAS

SEQ ID NO: 19 - protein, 14 aa, synthetic construct
WFRQAPGKEREFVA

SEQ ID NO: 20 - protein, 32 aa, synthetic construct
RFAISRDNAKNTVYLQMNSLKPEDTAVYYCAA

SEQ ID NO: 21 - protein, 118 aa, synthetic construct
QVQLVQSGGGLVQPGGSLRLSCAASGSISSNNVMGWYRQGPGKQREFVASITDSDLANYAASVKGRFTISRDNAKNTVNLQMNSLRPEDTDVYYCRLTMMTYPPTISWGQGTQVTVSS

SEQ ID NO: 22 - protein, 118 aa, synthetic construct
QVQLVQSGGGLVQPGGSLRLSCAASGSISSNNVMGWYRQGPGKQREFVASITDSDLANYAASVKGRFTISRDNAKNTVNLQMNSLRPEDTDVYYCRLTMMTYPPIISWGQGTQVTVSS

SEQ ID NO: 23 - protein, 118 aa, synthetic construct
QVQLVESGGGLVQPGGSLRLSCAASGTISSNNVMGWYRQGPGKQREFVASVTDSDLANYAASVKGRFTISRDNAKNTVNLQMNSLRPEDTGVYYCRLTIMTYPPTISWGQGTQVTVSS

SEQ ID NO: 24 - protein, 118 aa, synthetic construct
QVQLVQSGGGLVQPGGSLRLSCAASGSISSNNVMGWYRQGPGKQREFVASITDSDLANYAASVKGRFTISRDNAKNTVNLQMNSLRPEDTDVYYCRLTMMTAPPTISWGQGTQVTVSS

SEQ ID NO: 25 - protein, 118 aa, synthetic construct
QVQLVQSGGGLVQPGGSLRLSCAASGSISSNNVMGWYRQGPGKQREFVASITDSDLANYAASVKGRFTISRDNAKNTVNLQMNSLRPEDTDVYYCRLTMMTYAATISWGQGTQVTVSS

SEQ ID NO: 26 - protein, 118 aa, synthetic construct
QVQLVQSGGGLVQPGGSLRLSCAASGSISSNNVMGWYRQGPGKQREFVASITDSDLANYAASVKGRFTISRDNAKNTVNLQMNSLRPEDTDVYYCRLTMMTAAATISWGQGTQVTVSS

SEQ ID NO: 27 - protein, 121 aa, synthetic construct
QVQLVQSGGGLVQPGGSLRLSCAASGSISSNNVMGWYRQGPGKQREFVASITDSDLANYAASVKGRFTISRDNAKNTVNLQMNSLRPEDTDVYYCNANLRPRRWSAWQNYWGQGTQVTVSS

SEQ ID NO: 28 - protein, 123 aa, synthetic construct
QVQLVESGGGLVQAGGSLRLSCAASGVSFSRTSLGWFRQAPGKEREFVSVISWIGGTTYYADSLKGRFTISRDNAKSTVYLQMNSLKPEDTALYYCAISNGVLPRTEGAFASWGQGTQVTVSS

SEQ ID NO: 29 - protein, 123 aa, synthetic construct
QVQLVQSGGGLVQAGGSLRLSCAASGVSFSRTSLGWFRQAPGKEREFVSVISWIGGTTYYADSLKGRFTISRDNAKSTVYLQMNSLKPEDTALYYCAISNGVLPATAGAAASWGQGTQVTVSS

SEQ ID NO: 30 - protein, 123 aa, synthetic construct
QVQLVQSGGGLVQAGGSLRLSCAASGVSFSRTSLGWFRQAPGKEREFVSVISWIGGTTYYADSLKGRFTISRDNAKSTVYLQMNSLKPEDTAVYYCAARPAEQYGSLRPYNYWGQGTQVTVSS

SEQ ID NO: 31 - protein, 127 aa, synthetic construct
QVQLVQSGGGLVQAGDSLRLACAASEGTFRTNVMGWFRQAPGKEREFVAAISWSGGITHYSNSVKGRFAISRDNAKNTVYLQMNSLKPEDTAVYYCAADPSTTPWNLRVRVNEYEYWGQGTQVTVSS

SEQ ID NO: 32 - protein, 127 aa, synthetic construct
QVQLVQSGGGLVQAGDSLRLACAASEGTFRTNVMGWFRQAPGKEREFVAAISWSGGITHYSNSVKGRFAISRDNAKNTVYLQMNSLKPEDTAVYYCAADPSTTAAALRVRVNEYEYWGQGTQVTVSS

SEQ ID NO: 33 - protein, 127 aa, synthetic construct
QVQLVQSGGGLVQAGDSLRLACAASEGTFRTNVMGWFRQAPGKEREFVAAISWSGGITHYSNSVKGRFAISRDNAKNTVYLQMNSLKPEDTAVYYCAADPSTTPWNLAAAVNEYEYWGQGTQVTVSS

SEQ ID NO: 34 - protein, 128 aa, synthetic construct
QVQLVQSGGGLVQAGDSLRLACAASEGTFRTNVMGWFRQAPGKEREFVAAISWSGGITHYSNSVKGRFAISRDNAKNTVYLQMNSLKPEDTAVYYCAADRRVGATPNSYFDAKFYDPWGQGTQVTVSS

SEQ ID NO: 35 - protein, 10 aa, synthetic construct
GSISSNNVMG

SEQ ID NO: 36 - protein, 10 aa, synthetic construct
GTISSNNVMG

SEQ ID NO: 37 - protein, 16 aa, synthetic construct
SITDSDLANYAASVKG

SEQ ID NO: 38 - protein, 16 aa, synthetic construct
SVTDSDLANYAASVKG

SEQ ID NO: 39 - protein, 10 aa, synthetic construct
TMMTYPPTIS

SEQ ID NO: 40 - protein, 10 aa, synthetic construct
TMMTYPPIIS

SEQ ID NO: 41 - protein, 10 aa, synthetic construct
TIMTYPPTIS

SEQ ID NO: 42 - protein, 10 aa, synthetic construct
TMMTAPPTIS

SEQ ID NO: 43 - protein, 10 aa, synthetic construct
TMMTYAATIS

SEQ ID NO: 44 - protein, 10 aa, synthetic construct
TMMTAAATIS

SEQ ID NO: 45 - protein, 13 aa, synthetic construct
NLRPRRWSAWQNY

SEQ ID NO: 46 - protein, 14 aa, synthetic construct
RPAEQYGSLRPYNY

SEQ ID NO: 47 - protein, 18 aa, synthetic construct
DPSTTPWNLRVRVNEYEY

SEQ ID NO: 48 - protein, 18 aa, synthetic construct
DPSTTAAALRVRVNEYEY

SEQ ID NO: 49 - protein, 18 aa, synthetic construct
DPSTTPWNLAAAVNEYEY

SEQ ID NO: 50 - protein, 18 aa, synthetic construct
DRRVGATPNSYFDAKYDP

SEQ ID NO: 51 - protein, 781 aa, Homo sapiens
MATQADLMELDMAMEPDRKAAVSHWQQQSYLDSGIHSGATTTAPSLSGKGNPEEEDVDTSQVLYEWEQGFSQSFTQEQVADIDGQYAMTRAQRVRAAMFPETLDEGMQIPSTQFDAAHPTNVQRLAEPSQMLKHAVVNLINYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSNKPAIVEAGGMQALGLHLTDPSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDVHNRIVIRGLNTIPLFVQLLYSPIENIQRVAAGVLCELAQDKEAAEAIEAEGATAPLTELLHSRNEGVATYAAAVLFRMSEDKPQDYKKRLSVELTSSLFRTEPMAWNETADLGLDIGAQGEPLGYRQDDPSYRSFHSGGYGQDALGMDPMMEHEMGGHHPGADYPVDGLPDLGHAQDLMDGLPPGDSNQLAWFDTDL

SEQ ID NO: 52 - DNA, 28 bp, synthetic construct
  source 1..28: mol_type = other DNA
saggtgcagctggtrgagtctgggggag

SEQ ID NO: 53 - DNA, 23 bp, synthetic construct
  source 1..23: mol_type = other DNA
ggtacgtgctgttgaactgttcc

SEQ ID NO: 54 - protein, 59 aa, synthetic construct
GGSSTASGSGSGGSGTAGSSGGAGSSGGSTTAGGSASGSGSTGSGTGGASSGGASGASG

SEQ ID NO: 55 - protein, 217 aa, synthetic construct
APELLGGPSVFIFPPKPKDVLSISGRPEVTCVVVDVGQEDPEVSFNWYIDGAEVRTANTRPKEEQFNSTYRVVSVLPIQHQDWLTGKEFKCKVNNKALPAPIEKTISKAKGQTREPQVYTLAPHREELAKDTVSVTCLVKGFYPPDINVEWQRNRQPEPEGTYATTPPQLDNDGTYFLYSKLSVGKNTWQRGETFTCVVMHEALHNHYTQKSITQSS

SEQ ID NO: 56 - protein, 217 aa, synthetic construct
GPELLGGPTVFIFPPKPKDVLSITRKPEVTCVVVDVGKEDPEIEFSWSVDDTEVHTAETKPKEEQFNSTYRVVSVLPIQHQDWLTGKEFKCKVNNKALPAPIERTISKAKGQTREPQVYTLAPHREELAKDTVSVTCLVKGFFPADINVEWQRNGQPESEGTYATTPPQLDNDGTYFLYSKLSVGKNTWQQGEVFTCVVMHEALHNHSTQKSISQSP

SEQ ID NO: 57 - protein, 15 aa, synthetic construct
AHHSEDPSSKCPKCP

SEQ ID NO: 58 - protein, 29 aa, synthetic construct
EPKTPKPQPQPQPQPQPNPTTESKCPKCP

SEQ ID NO: 59 - protein, 335 aa, synthetic construct
QVQLVQSGGGLVQPGGSLRLSCAASGSISSNNVMGWYRQGPGKQREFVASITDSDLANYAASVKGRFTISRDNAKNTVNLQMNSLRPEDTDVYYCRLTMMTYPPTISWGQGTQVTVSSAPELLGGPSVFIFPPKPKDVLSISGRPEVTCVVVDVGQEDPEVSFNWYIDGAEVRTANTRPKEEQFNSTYRVVSVLPIQHQDWLTGKEFKCKVNNKALPAPIEKTISKAKGQTREPQVYTLAPHREELAKDTVSVTCLVKGFYPPDINVEWQRNRQPEPEGTYATTPPQLDNDGTYFLYSKLSVGKNTWQRGETFTCVVMHEALHNHYTQKSITQSS

SEQ ID NO: 60 - protein, 344 aa, synthetic construct
QVQLVQSGGGLVQAGDSLRLACAASEGTFRTNVMGWFRQAPGKEREFVAAISWSGGITHYSNSVKGRFAISRDNAKNTVYLQMNSLKPEDTAVYYCAADPSTTPWNLRVRVNEYEYWGQGTQVTVSSGPELLGGPTVFIFPPKPKDVLSITRKPEVTCVVVDVGKEDPEIEFSWSVDDTEVHTAETKPKEEQFNSTYRVVSVLPIQHQDWLTGKEFKCKVNNKALPAPIERTISKAKGQTREPQVYTLAPHREELAKDTVSVTCLVKGFFPADINVEWQRNGQPESEGTYATTPPQLDNDGTYFLYSKLSVGKNTWQQGEVFTCVVMHEALHNHSTQKSISQSP

SEQ ID NO: 61 - protein, 350 aa, synthetic construct
QVQLVQSGGGLVQPGGSLRLSCAASGSISSNNVMGWYRQGPGKQREFVASITDSDLANYAASVKGRFTISRDNAKNTVNLQMNSLRPEDTDVYYCRLTMMTYPPTISWGQGTQVTVSSAHHSEDPSSKCPKCPAPELLGGPSVFIFPPKPKDVLSISGRPEVTCVVVDVGQEDPEVSFNWYIDGAEVRTANTRPKEEQFNSTYRVVSVLPIQHQDWLTGKEFKCKVNNKALPAPIEKTISKAKGQTREPQVYTLAPHREELAKDTVSVTCLVKGFYPPDINVEWQRNRQPEPEGTYATTPPQLDNDGTYFLYSKLSVGKNTWQRGETFTCVVMHEALHNHYTQKSITQSS

SEQ ID NO: 62 - protein, 373 aa, synthetic construct
QVQLVQSGGGLVQAGDSLRLACAASEGTFRTNVMGWFRQAPGKEREFVAAISWSGGITHYSNSVKGRFAISRDNAKNTVYLQMNSLKPEDTAVYYCAADPSTTPWNLRVRVNEYEYWGQGTQVTVSSEPKTPKPQPQPQPQPQPNPTTESKCPKCPGPELLGGPTVFIFPPKPKDVLSITRKPEVTCVVVDVGKEDPEIEFSWSVDDTEVHTAETKPKEEQFNSTYRVVSVLPIQHQDWLTGKEFKCKVNNKALPAPIERTISKAKGQTREPQVYTLAPHREELAKDTVSVTCLVKGFFPADINVEWQRNGQPESEGTYATTPPQLDNDGTYFLYSKLSVGKNTWQQGEVFTCVVMHEALHNHSTQKSISQSP

(19) United States
(12) Patent Application Publication
(10) Pub. No.: US 2026/0167713 A1
(43) Pub. Date: Jun. 18, 2026

(54) ANTI-BETA-CATENIN ANTIBODIES

(71) Applicant: UCB BIOPHARMA SRL, Brussels (BE)

(72) Inventors: Alastair David Griffiths Lawson, Slough (GB); Laura Starkie, Slough (GB); MIchael John Wright, Slough (GB)

(21) Appl. No.: 19/129,397

(22) PCT Filed: Nov. 16, 2023
(86) PCT No.: PCT/EP2023/082134

§ 371 Date: May 13, 2025

(30) Foreign Application Priority Data
Nov. 18, 2022 (GB) 2217301.7

(51) Int. Cl. C07K 16/28

(52) U.S. Cl. CPC C07K 16/28; C07K 2317/569; C07K 2317/76; C07K 2317/92

(57) ABSTRACT

The present disclosure relates to antibodies directed against betacatenin and formulations comprising the same. The disclosure further relates to the use of the beta-catenin antibodies and formulations in therapy, notably in the treatment of solid cancers.

### FIELD OF THE INVENTION

The present invention relates to antibodies directed against beta-catenin and formulations comprising the same. The invention further relates to the use of the beta-catenin antibodies and formulations in therapy, notably in the treatment of solid cancers.

### BACKGROUND OF THE INVENTION

Intracellular antibodies (also named intrabodies) are antibody or antibody fragments that are located within a cell, sometimes in a particular intracellular compartment by virtue of intracellular trafficking signals, where they interact with the target antigen. To function as intrabodies, the antibodies must retain stability and affinity for the target in the reducing environment of the cell. Full length antibodies are inherently unstable inside the cell, because antibodies rely on inter and intra-chain disulphide bonds for stability, which cannot form inside the reducing environment of the cell cytoplasm (Biocca et al., 1995). Nonetheless there are other stable options, including the use of engineered antibody fragments or alternative non-immunoglobulin binding proteins. Intrabodies have demonstrated utility as research tools for modulating intracellular target protein function (Stocks, 2005).

Camelidae are known to produce Heavy-Chain Antibodies (HCAbs) in addition to conventional heavy and light chain containing antibodies, as part of their adaptive immune response. As these HCAbs lack both the entire light chain and the CH1 domains of conventional IgGs, the antigen binding is therefore mediated entirely through the unpaired VH domain, referred to as a VHH. There were several reports of llama VHH intrabodies which function intracellularly (Serruys et al., 2009; Vercruysse et al., 2010). VHH domains have already been used as research intrabodies due to their intrinsic domain stability and low aggregation.

Wnt proteins are a large family of lipid-modified secreted signalling proteins, defined by amino acid sequences rather than functional properties. They are highly conserved across the animal kingdom. Wnt signalling is initiated by the binding of Wnt lipoproteins to extracellular receptors, such as the 7-transmembrane Frizzled receptor family, LRP5 and six co-receptors, and the receptor tyrosine kinases Ryk and ROR. Studies of beta-catenin have demonstrated that it has at least two cellular roles, one of them being the key transcriptional co-activator in the canonical Wnt signalling pathway and the second in cell-cell adhesion, as an important component of the adherens junction, where it links E-cadherin to the cell cytoskeleton via alpha-catenin (Ben-Ze'ev and Geiger, 1998). Activation of the canonical Wnt/b-catenin pathway by exogenous Wnt results in the stabilization and activation of beta-catenin, which translocates from the cytoplasm into the nucleus where it interacts with numerous partners including the TCF/LEF family. Formation of the bipartite beta-catenin/TCF transcription factor2 activates the transcription of Wnt responsive genes.

Aberrant Wnt signalling pathway is implicated in many diverse diseases, including colorectal cancers, adenomatous polyposis (FAP), colon cancer, melanoma, hepatocellular carcinoma, ovarian cancer, endometrial cancer, medulloblastoma pilomatricomas, and prostate cancer as well as bone density disorders, Alzheimer's disease (Morin et al., 1999; Newnham et al., 2015) schizophrenia (Miyaoka et al., 1999), type II diabetes (Grant et al., 2006), rheumatoid arthritis (Sen et al., 2000), oligodentonia (Lammi et al., 2004) osteoporosis-pseudoglioma syndrome (Gong et al., 2001); familial exudative vitreoretinopathy (Toomes et al., 2004) or yet vascular calcification. As a non-limiting example, the majority of colorectal cancers have a mutation in the Wnt signalling pathway, particularly in the APC gene (Bienz and Clevers, 2000), which result in the stabilisation and excessive transcriptional activity of beta-catenin.

Some antibodies targeting beta-catenin have already been disclosed. For instance, McCrea (1993) described a Fab moiety, directed to the N-term residues 6-138 of beta-catenin, capable of influencing the developmental pattern in Xenopus embryos. WO2021015419 discloses an antibody that specifically binds to phosphorylated beta-catenin and has its epitope composed of amino acid residues 42-51 of the full-length beta-catenin.

Therefore, there remains a need to provide antibodies which inhibit Wnt signalling by binding and blocking beta-catenin biological activities such as its transcriptional activating activity. Such antibodies would be very useful in therapy, including for therapeutic intervention in colorectal cancer or adenomatous polyposis. In particular it remains the need to provide anti beta-catenin antibodies which function inside the cell, as intrabodies, to disrupt the transcriptional activating activity of beta-catenin.

### SUMMARY OF THE INVENTION

The present invention addresses the above-identified need by providing new antibodies against beta-catenin. These antibodies can be useful in therapy, notably in the treatment of colorectal cancer or adenomatous polyposis. These antibodies have notably high specificity for beta-catenin and specifically disrupts beta-catenin co-transcriptional activity without affecting its role at the plasma membrane.

In a first aspect, the present invention provides an antibody that specifically binds to human beta-catenin, wherein said antibody comprises a heavy chain variable region comprising: i) a CDR-H1 comprising SEQ ID NO: 1, 35 or 36; a CDR-H2 comprising SEQ ID NO: 2, 37 or 38; and a CDR-H3 comprising SEQ ID NO: 3, 39, 40, 41, 42, 43, 44 or 45; ii) a CDR-H1 comprising SEQ ID NO: 4; a CDR-H2 comprising SEQ ID NO: 5 and a CDR-H3 comprising SEQ ID NO: 6 or 46; iii) a CDR-H1 comprising SEQ ID NO: 7; a CDR-H2 comprising SEQ ID NO: 8 and a CDR-H3 comprising SEQ ID NO: 9, 47, 48, 49 or 50; or iv) CDR-H1, CDR-H2 and CDR-H3 sequences that have at least 70%, 80%, 90%, 91%, 92%, 93%, 94%, 95%, 96%, 97%, 98%, or 99% identity or similarity to any of the sequences listed in any one of i. to iii. Preferably, the antibody is a VHH.

In a second aspect, the present invention provides an antibody that specifically binds to human beta-catenin, wherein said antibody comprises framework regions (FRs) comprising or consisting of: i) a FR1 comprising SEQ ID NO: 10, 15, or 18 or a sequence that has at least 70%, 80%, 90%, 91%, 92%, 93%, 94%, 95%, 96%, 97%, 98%, or 99% identity thereof; ii) a FR2 comprising SEQ ID NO: 11, 16 or 19 or a sequence that has at least 70%, 80%, 90%, 91%, 92%, 93%, 94%, 95%, 96%, 97%, 98%, or 99% identity thereof; iii) a FR3 comprising SEQ ID NO: 12, 13, 17 or 20 or a sequence that has at least 70%, 80%, 90%, 91%, 92%, 93%, 94%, 95%, 96%, 97%, 98%, or 99% identity thereof; and iv) a FR4 comprising SEQ ID NO: 14 or a sequence that has at least 70%, 80%, 90%, 91%, 92%, 93%, 94%, 95%, 96%, 97%, 98%, or 99% identity thereof. Preferably, the antibody is a VHH.

In a third aspect, the present invention provides an antibody that specifically binds to human beta-catenin, wherein the antibody has a heavy chain variable region comprising any one of SEQ ID NO: 21-25, 28, 31 or 33 or a sequence that has at least 70%, 80%, 90%, 91%, 92%, 93%, 94%, 95%, 96%, 97%, 98%, or 99% identity thereof.

In further aspects of the invention are covered isolated polynucleotides encoding the antibody, cloning or expression vectors, host cells, processes for the production of the antibody, pharmaceutical compositions comprising the antibody, and their use in therapy.

### DETAILED DESCRIPTION OF THE INVENTION

The present disclosure will now be described with respect to particular non-limiting aspects and embodiments thereof and with reference to certain figures and examples.

Unless defined otherwise, all technical and scientific terms used herein have the same meaning as is commonly understood by one of skill in art to which the subject matter herein belongs. As used in the specification and claims, the following definitions are supplied to facilitate the understanding of the present invention.
The term “and/or” used in a phrase such as “A and/or B” herein is intended to include “A and B”, “A or B”, “A”, and “B”.
The forms “a”, “an”, and “the” include both single and plural referents unless the content clearly dictates otherwise. Thus, for example, reference to “an antibody” includes both “an antibody” and “antibodies”.
The term “comprising” does not exclude other elements. For the purpose of the present disclosure, the term “consisting of” is considered to be a preferred embodiment of the term “comprising of”.
The term “beta-catenin” refers to a polypeptide involved in, among other functions, the Wnt pathway and cell to cell adhesion. The amino acid and nucleic sequences of beta-catenin and its isoforms are also well known in the art (see e.g. UNIPROT P35222). The full human beta-catenin comprises the sequence given in SEQ ID NO: 51. The term also covers any alternative splicing or natural variants of human beta-catenin which are naturally expressed by cells.
The term “antibody” refers to whole antibodies and functionally active fragments thereof (i.e., molecules that contain an antigen binding domain that specifically binds an antigen, also termed antigen-binding fragments). Features described herein with respect to antibodies also apply to antibody fragments unless context dictates otherwise. Whole antibodies, also known as “immunoglobulins (Ig)” generally relate to intact or full-length antibodies i.e. comprising the elements of two heavy chains and two light chains, inter-connected by disulphide bonds, which assemble to define a characteristic Y-shaped three-dimensional structure. Classical natural whole antibodies are monospecific in that they bind one antigen type, and bivalent in that they have two independent antigen binding domains.
The terms “intact antibody”, “full-length antibody” and “whole antibody” are used interchangeably to refer to a monospecific bivalent antibody having a structure similar to a native antibody structure, including an Fc region as defined herein. In whole antibodies, each light chain is comprised of a light chain variable region (abbreviated herein as VL) and a light chain constant region (CL). Each heavy chain is comprised of a heavy chain variable region (abbreviated herein as VH) and a heavy chain constant region (CH) constituted of three constant domains CH1, CH2 and CH3, or four constant domains CH1, CH2, CH3 and CH4, depending on the Ig class. The “class” of an Ig or antibody refers to the type of constant region and includes IgA, IgD, IgE, IgG and IgM and several of them can be further divided into subclasses, e.g. IgG1, IgG2, IgG3, IgG4. The constant regions of the antibodies may mediate the binding of the immunoglobulin to host tissues or factors, including various cells of the immune system (e.g., effector cells) and the first component (Clq) of the classical complement system. The VH and VL regions of the antibody according to the present invention can be further subdivided into regions of hypervariability (or “hypervariable regions”, or HVR) determining the recognition of the antigen, termed complementarity determining regions (CDR), interspersed with regions that are more structurally conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The CDRs and the FR together form a variable region. By convention, the CDRs in the heavy chain variable region of an antibody or antigen-binding fragment thereof are referred as CDR-H1, CDR-H2 and CDR-H3 and in the light chain variable regions as CDR-L1, CDR-L2 and CDR-L3. They are numbered sequentially in the direction from the N-terminus to the C-terminus of each chain. CDRs are conventionally numbered according to a system devised by Kabat et al. This system is set forth in Kabat et al., 1991. This numbering system is used in the present specification except where otherwise indicated.

The CDRs of the heavy chain variable domain typically comprise residues 31-35 (CDR-H1), residues 50-65 (CDR-H2) and residues 95-102 (CDR-H3) according to the Kabat numbering system. However, according to Chothia (1987), the loop equivalent to CDR-H1 extends from residue 26 to residue 32. Thus, unless indicated otherwise ‘CDR-H1’ as employed herein is intended to refer to residues 26 to 35, as described by a combination of the Kabat numbering system and Chothia's topological loop definition. The CDRs of the light chain variable domain are typically located at residues 24-34 (CDR-L1), residues 50-56 (CDR-L2) and residues 89-97 (CDR-L3) according to the Kabat numbering system. In addition to the CDR loops, a fourth loop exists between CDR-2 (CDR-L2 or CDR-H2) and CDR-3 (CDR-L3 or CDR-H3) which is formed by framework 3 (FR3). The Kabat numbering system defines framework 3 as positions 66-94 in a heavy chain and positions 57-88 in a light chain.
The terms “constant domain(s)” or “constant region”, as used herein, are used interchangeably to refer to the domain(s) of an antibody which is outside the variable regions. The constant domains are identical in all antibodies of the same isotype but are different from one isotype to another.

Typically, the constant region of a heavy chain is formed, from N to C terminal, by CH1-hinge-CH2-CH3-optionally CH4, comprising three or four constant domains.

The constant region domains of the antibody molecule of the present invention, if present, may be selected having regard to the proposed function of the antibody molecule, and in particular the effector functions which may be required. For example, the constant region domains may be human IgA, IgD, IgE, IgG or IgM domains. In particular, human IgG constant region domains may be used, especially of the IgG1 and IgG3 isotypes when the antibody molecule is intended for therapeutic uses and antibody effector functions are required. Alternatively, IgG2 and IgG4 isotypes may be used when the antibody molecule is intended for therapeutic purposes and antibody effector functions are not required. It will be appreciated that sequence variants of these constant region domains may also be used. For example, IgG4 molecules in which the serine at position 241 (numbered according to the Kabat numbering system) has been changed to proline as described in Angal et al. (Angal et al., 1993).
“Fc”, “Fc fragment”, and “Fc region” are used interchangeably to refer to the C-terminal region of an antibody comprising the constant region of an antibody excluding the first constant region immunoglobulin domain. Thus, Fc refers to the last two constant domains, CH2 and CH3, of IgA, IgD, and IgG, or the last three constant domains of IgE and IgM, and the flexible hinge N-terminal to these domains. The human IgG1 heavy chain Fc region is defined herein to comprise residues C226 to its carboxyl-terminus, wherein the numbering is according to the EU index as in Kabat. In the context of human IgG1, the lower hinge refers to positions 226-236, the CH2 domain refers to positions 237-340 and the CH3 domain refers to positions 341-447 according to the EU index as in Kabat. The corresponding Fc region of other immunoglobulins can be identified by sequence alignments. In the context of the present disclosure, when present, the constant region or Fc region may be natural, as defined above, or else may be modified in various ways, provided that it comprises a functional FcR binding domain, and preferably a functional FcRn binding domain.
The term “antibody” encompasses monovalent antibodies, i.e. antibodies comprising only one antigen binding domain (e.g. one-armed antibodies comprising a full-length heavy chain and a full-length light chain interconnected, also termed “half-antibody”), and multivalent antibodies, or antibodies comprising more than one antigen binding domain.
The term “antibody” according to the invention also encompasses antigen-binding fragments of antibodies. Antigen-binding fragments of antibodies include single chain antibodies (e.g. scFv, and dsscfv), Fab, Fab′, F(ab′)₂, Fv, single domain antibodies or nanobodies (e.g. VH or VL, or VHH or VNAR).
The term “Fab fragment” as used herein refers to an antibody fragment comprising a light chain fragment comprising a VL (variable light) domain and a constant domain of a light chain (CL), and a VH (variable heavy) domain and a first constant domain (CH1) of a heavy chain. A typical “Fab′ fragment” comprises a heavy and a light chain pair in which the heavy chain comprises a variable region VH, a constant domain CH1 and a natural or modified hinge region and the light chain comprises a variable region VL and a constant domain CL. Dimers of a Fab′ according to the present disclosure create a F(ab′)₂ where, for example, dimerization may be through the hinge.
The term “single domain antibody” as used herein refers to an antibody fragment consisting of a single monomeric variable antibody domain. Examples of single domain antibodies include VH, VL, VHH or VNAR.
The term “HCab” refers to an antibody which comprises or consists of a VHH domain linked directly or via a linker (hinge) to a CH2 and a CH3 domains.
The term “Fv” refers to two variable domains, for example co-operative variable domains, such as a cognate pair or affinity matured variable domains, i.e. a VH and VL pair.
The terms “Single chain variable fragment” or “scFv” as employed herein refer to a single chain variable fragment which is stabilised by a peptide linker between the VH and VL variable domains.
The terms “multispecific” or “Multi-specific antibody” as employed herein refer to an antibody as described herein which has at least two binding domains, i.e. two or more binding domains, for example two or three binding domains, wherein the at least two binding domains independently bind two different antigens or two different epitopes on the same antigen. Multi-specific antibodies are generally monovalent for each specificity (antigen). Multi-specific antibodies described herein encompass monovalent and multivalent, e.g. bivalent, trivalent, tetravalent multi-specific antibodies.
The term “antigen binding domain” as employed herein refers to a portion of the antibody, which comprises a part or the whole of one or more variable domains, for example a part or the whole of a pair of variable domains VH and VL, that interacts specifically with the target antigen. A binding domain may comprise a single domain antibody. In one embodiment, each binding domain is monovalent. Preferably each binding domain comprises no more than one VH and one VL.
The term “chimeric” antibody refers to an antibody in which the variable domain (or at least a portion thereof) of the heavy and/or light chain is derived from a particular source or species, for example a mouse, rat, rabbit or similar while the remainder of the heavy and/or light chain (i.e. the constant region) is derived from another species such as a human. Chimeric antibodies are composed of elements derived from two different species such that the element retains the characteristics of the species from which it is derived. A subcategory of “chimeric antibodies” is “humanized antibodies”. Humanised antibodies (which include CDR-grafted antibodies) are antibody molecules having one or more complementarity determining regions (CDRs) from a non-human species and a framework region from a human immunoglobulin molecule. It will be appreciated that it may only be necessary to transfer the specificity determining residues of the CDRs rather than the entire CDR (Kashmiri et al., 2005, Methods, 36, 25-34). Humanised antibodies may optionally further comprise one or more framework residues derived from the non-human species from which the CDRs were derived.
The term “Fully human” antibodies refers to antibodies in which the variable regions and the constant regions (where present) of both the heavy and the light chains are all of human origin, or substantially identical to sequences of human origin, but not necessarily from the same antibody.

Examples of fully human antibodies may include antibodies produced, for example by the phage display methods described above and antibodies produced by mice in which the murine immunoglobulin variable and optionally the constant region genes have been replaced by their human counterparts.
Within the present invention, the term “epitope” is used interchangeably for both conformational and linear epitopes. A conformational epitope is composed of discontinued sections of the antigen's amino acid primary sequence and a linear epitope is formed by a sequence formed by continuous amino acids.
The term “isolated” antibody refers to an antibody which has been separated (e.g. by purification means) from a component of its natural environment.
The term “isolated” polynucleotide means that the polynucleotide exists in a physical milieu distinct from that in which it may occur in nature.
The term “K_{D}” as used herein refers to the equilibrium dissociation constant which is obtained from the ratio of K_{d} to Kₐ (i.e. Kₐ/Kₐ) and is expressed as a molar concentration (M). Kₐ and Kₐ refers to the dissociation rate and association rate, respectively, of a particular antigen-antibody (or antigen-binding fragment thereof) interaction. K_{D} values for antibodies can be determined using methods well established in the art, such as (but not limited to) those used in the example section.
The term “blocking”, “blocks” and the like, in the context of antibodies describe an antibody that is capable of inhibiting or attenuating at least one of the biological activities of its target (beta-catenin). Alternatively, the term “neutralizing” or neutralizes” can be used.
The term “specifically binds” refers to an antibody which binds with preferential or high affinity to the protein of interest (e.g. beta-catenin) but does not substantially bind to other proteins. In other words, the antibody binds to the protein of interest with no significant cross-reactivity to any other molecule. The specificity of an antibody may be further studied by determining whether or not the antibody binds to other related proteins as discussed above or whether it discriminates between them.
As used herein, the terms “treatment”, “treating” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” thus covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.
The term “therapeutically effective amount” refers to the amount of an active ingredient (such as the antibodies according to the invention) that, when administered to a mammal or other subject for treating a disease, is sufficient to produce such treatment for the disease.

There were several challenges associated to the provision of antibodies against beta-catenin such as: 1) the fact that beta-catenin is an intracellular protein and thus that any anti-beta-catenin antibody administered in vivo would have to cross the cell membrane, but also 2) because the target (i.e. beta-catenin) has two major but completely different activities and it may be valuable to block only one of these biological activities. In particular, as it will now be described in more detail, the invention is based on the finding of antagonistic anti-beta-catenin antibodies which can neutralise/inhibit beta-catenin transcriptional activating activity without impacting its activity in cell-to-cell adhesion.

The present invention also provide evidence that the epitope recognised by the antibodies of the invention is found in the natural conformation(s) of intra cellular beta-catenin, thereby supporting for the first time the use of an anti-beta-catenin antibody being in a VHH format and able to act as an intrabody.

The main object of the present invention is an antibody that specifically binds to beta-catenin, wherein the antibody comprises a heavy chain variable region comprising:
i. a CDR-H1 of SEQ ID NO: 1, 35 or 36; a CDR-H2 of SEQ ID NO: 2, 37 or 38; and a CDR-H3 of SEQ ID NO: 3, 39, 40, 41, 42, 43, 44 or 45;
ii. a CDR-H1 of SEQ ID NO: 4; a CDR-H2 of SEQ ID NO: 5 and a CDR-H3 of SEQ ID NO: 6 or 46;
iii. a CDR-H1 of SEQ ID NO: 7; a CDR-H2 of SEQ ID NO: 8 and a CDR-H3 of SEQ ID NO: 9, 47, 48, 49 or 50; or iv. CDR-H1, CDR-H2 and CDR-H3 sequences that have at least 70%, 80%, 90%, 91%, 92%, 93%, 94%, 95%, 96%, 97%, 98%, or 99% identity or similarity to any of the sequences listed in any one of i. to iii.

As non-limiting examples, the antibody according to the invention can be: a) an antibody which comprises a CDR-H1 according to SEQ ID NO: 1; a CDR-H2 according to SEQ ID NO: 2 and a CDR-H3 according to SEQ ID NO: 3, b) an antibody which comprises a CDR-H1 according to SEQ ID NO: 4; a CDR-H2 according to SEQ ID NO: 5 and a CDR-H3 according to SEQ ID NO: 6, c) an antibody which comprises a CDR-H1 according to SEQ ID NO: 7; a CDR-H2 according to SEQ ID NO: 8 and a CDR-H3 according to SEQ ID NO: 9, d) the antibody “V1” which comprises a CDR-H1 according to SEQ ID NO: 35; a CDR-H2 according to SEQ ID NO: 37 and a CDR-H3 according to SEQ ID NO: 39; e) the antibody “V2” which comprises a CDR-H1 according to SEQ ID NO: 35; a CDR-H2 according to SEQ ID NO: 37 and a CDR-H3 according to SEQ ID NO: 40, f) the antibody “V3” which comprises a CDR-H1 according to SEQ ID NO: 36; a CDR-H2 according to SEQ ID NO: 38 and a CDR-H3 according to SEQ ID NO: 41, g) the antibody “S” which comprises a CDR-H1 according to SEQ ID NO: 4; a CDR-H2 according to SEQ ID NO: 5 and a CDR-H3 according to SEQ ID NO: 6 or yet h) the antibody “O” which comprises a CDR-H1 according to SEQ ID NO: 7; a CDR-H2 according to SEQ ID NO: 8 and a CDR-H3 according to SEQ ID NO: 47.

The anti-beta-catenin antibodies according to the invention (i.e. comprising any one of the above combinations of CDR sequences) are particularly inventive because they provide for an antibody with high affinity for human beta-catenin, high inhibition for at least one beta-catenin biological functions (preferably the function in the Wnt pathway) and high stability which is essential for manufacturability.

In one embodiment of the present invention, the antibody further comprises framework regions (FRs) comprising (or consisting of):
i. A FR1 comprising SEQ ID NO: 10, 15, or 18 or a sequence that has at least 70%, 80%, 90%, 91%, 92%, 93%, 94%, 95%, 96%, 97%, 98%, or 99% identity thereof;
ii. A FR2 comprising SEQ ID NO: 11, 16 or 19 or a sequence that has at least 70%, 80%, 90%, 91%, 92%, 93%, 94%, 95%, 96%, 97%, 98%, or 99% identity thereof;
iii. A FR3 comprising SEQ ID NO: 12, 13, 17 or 20 or a sequence that has at least 70%, 80%, 90%, 91%, 92%, 93%, 94%, 95%, 96%, 97%, 98%, or 99% identity thereof; and
iv. A FR4 comprising SEQ ID NO: 14 or a sequence that has at least 70%, 80%, 90%, 91%, 92%, 93%, 94%, 95%, 96%, 97%, 98%, or 99% identity thereof.

In another embodiment, the present invention provides an antibody that specifically binds to beta-catenin, wherein the antibody has a heavy chain variable region comprising any one of SEQ ID NO: 21, 22, 23, 24, 25, 28, 31 or 33, or a sequence that has at least 70%, 80%, 90%, 91%, 92%, 93%, 94%, 95%, 96%, 97%, 98%, or 99% identity thereof.

In another embodiment, the present invention provides an antibody that specifically binds to beta-catenin, wherein the antibody further comprises a CH2 and a CH3 domains. Non-limiting examples of CH2/CH3 domains comprise any one of SEQ ID Nos: 55 or 56, or a sequence that has at least 70%, 80%, 90%, 91%, 92%, 93%, 94%, 95%, 96%, 97%, 98%, or 99% identity thereof. The heavy chain variable region can be linked as such to the CH2/CH3 domains or can be linked via a linker (alternatively named a hinge). Non-limiting examples of linkers comprise any one of SEQ ID Nos: 57 or 58. Non-limiting examples of HCab comprising a heavy chain variable region and a CH2/CH3 domain with/without linkers can be selected among any one of SEQ ID Nos: 59, 60, 61 and 62 or a sequence that has at least 70%, 80%, 90%, 91%, 92%, 93%, 94%, 95%, 96%, 97%, 98%, or 99% identity thereof.

The antibody according to the invention as a whole is preferably a VHH or a Hcab, i.e. it is not associated to any light chains. In other words, the antibody according to the invention as a whole preferably consists of only one heavy chain variable domain and active fragments thereof or on only one full heavy chain and active fragments thereof. As it was shown that the antibodies according to the invention are able to be active intracellularly, they are alternatively called anti-beta-catenin intrabodies. The antibody herein described can be chimeric or humanized. Should the antibodies be humanised, suitable framework regions for the heavy chain of the humanized antibody according to the present invention can be derived from human germlines. Although they are preferably in a VHH format or Hcab format (even preferably in a VHH format), alternately they can be associated with light chains comprising CDRs that specifically bind to beta-catenin. In another alternative they can be associated with light chains comprising CDRs that specifically bind to one or more other targets in order to obtain a bispecific or multispecific antibody.

The anti-beta-catenin antibody according to the invention as a whole is an antagonistic antibody. Preferably it neutralises/inhibits at least one of the beta-catenin biological activities. Preferably the at least one beta-catenin biological activity that is neutralised/inhibited is its transcriptional activating activity. More preferably, only one beta-catenin biological activity is neutralised/inhibited, i.e. its transcriptional activating activity. As particular examples, the anti-beta-catenin antibodies described above i) block the binding of any one or more of Pontin52, Bcl-9 and lymphoid-enhancing factor-1 (LEF-1)/T-cell factor (TCF) family (TCF) to beta-catenin, and/or ii) neutralise or inhibit beta-catenin mediated pathway such as beta-catenin transcriptional activating activity. In another example, the anti-beta-catenin antibodies described herein prevent the interaction of any one or more of Pontin52, Bcl-9 and lymphoid-enhancing factor-1 (LEF-1)/T-cell factor (TCF) family (TCF) to beta-catenin, therefore inhibiting the beta-catenin transcriptional activating activity.

The anti-beta-catenin antibody according to the invention as a whole has preferably an equilibrium dissociation constant (KD) of 20 nM or less than 20 nM for beta-catenin, preferably 15 nM or less than 15 nM, even preferably 10 nM or less than 10 nM, such as 9 nM or less than 9 nM, 8 nM or less than 8 nM, 7 nM or less than 7 nM. In certain embodiments, the kD is as low as 6 nM or below, 5 nM or below or even 4 nM or below. The KD can be measured/determined by any standard methods. 5 For instance, the constant of dissociation can be determined by SPR at a temperature of 25° C., between an antibody of the invention and beta-catenin.

It has been shown that the anti-beta-catenin antibody according to the invention binds an epitope located between residues 138-390 of full human beta-catenin (as described in SEQ ID NO. 51). Therefore, herein described is an anti-beta-catenin antibody which specifically binds to human beta-catenin on an epitope located between residues 138-390 of SEQ ID NO. 51. Said epitope can be a conformational epitope or a linear epitope.

The epitope can be identified by any suitable epitope mapping method known in the art in combination with any one of the antibodies provided by the present invention. Examples of such methods include screening peptides of varying lengths derived from full length beta-catenin for binding to the antibody or fragment thereof of the present invention and identifying the smallest fragment that can specifically bind to the antibody containing the sequence of the epitope recognized by the antibody. beta-catenin peptides may be produced synthetically or by proteolytic digestion of the beta-catenin. Peptides that bind the antibody can be identified by, for example, mass spectrometric analysis. Methodologies such as X-ray crystallography, Nuclear magnetic resonance (NMR) spectroscopy or Hydrogen deuterium exchange mass spectrometry (HDX-MS) can be used to identify the epitope bound by an antibody. Typically, when the epitope determination is performed by X-ray crystallography, amino acid residues of the antigen within 4 Å from CDRs are considered to be amino acid residues part of the epitope. Once identified, the epitope may serve for preparing fragments which bind an antibody of the present invention and, if required, used as an immunogen to obtain additional antibodies which bind the same epitope.

The epitope as indicated in the aspects and embodiments describing the present invention is preferably an epitope characterized by X-ray crystallography. In one embodiment, the present invention provides an anti-beta-catenin antibody which binds to a conformational or linear epitope on beta-catenin, said conformational or linear epitope comprising at least 3, at least 4, at least 5, at least 6, at least 7, at least 8, at least 9, at least 10, in the region located from residues 164-390. In one embodiment of the invention, herein provided is an antibody that cross-competes for binding to beta-catenin with an antibody comprises heavy chain variable region comprising:
i. a CDR-H1 of SEQ ID NO: 1, 35 or 36; a CDR-H2 of SEQ ID NO: 2, 37 or 38; and a CDR-H3 of SEQ ID NO: 3, 39, 40, 41, 42, 43, 44 or 45;
ii. a CDR-H1 of SEQ ID NO: 4; a CDR-H2 of SEQ ID NO: 5 and a CDR-H3 of SEQ ID NO: 6 or 46;
iii. a CDR-H1 of SEQ ID NO: 7; a CDR-H2 of SEQ ID NO: 8 and a CDR-H3 of SEQ ID NO: 9, 47, 48, 49 or 50;
iv. a sequence according to any one of SEQ ID NO: 21-25, 28, 31 or 33;
V. a sequence according to any one of SEQ ID NO: 59, 60, 61 or 62;
vi. CDR-H1, CDR-H2 and CDR-H3 that have at least 70%, 80%, 90%, 91%, 92%, 93%, 94%, 95%, 96%, 97%, 98%, or 99% identity or similarity to any of the sequences listed in any one of i. to iii); or
vii. a sequence that has at least 70%, 80%, 90%, 91%, 92%, 93%, 94%, 95%, 96%, 97%, 98%, or 99% identity or similarity to any of the sequences listed in any one of iv. to v.

To determine if an antibody competes for binding with a reference antibody, the above-described binding methodology is performed in two different experimental setups. In a first setup, the reference antibody is allowed to bind to the antigen under saturating conditions followed by assessment of binding of the test antibody to the antigen. In a second setup, the test antibody is allowed to bind to the antigen under saturating conditions followed by assessment of binding of the reference antibody to the protein/peptide. If, in both experimental setups, only the first (saturating) antibody is capable of binding to the protein/peptide, then it is concluded that the test antibody and the reference antibody compete for binding to the antigen. As will be appreciated by the skilled person, an antibody that competes for binding with a reference antibody may not necessarily bind to the identical epitope as the reference antibody, but may sterically block binding of the reference antibody by binding an overlapping or adjacent epitope or cause a conformational change leading to the lack of binding.

Two antibodies bind to the same or overlapping epitope if each competitively inhibits (blocks) binding of the other to the antigen. Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other. Additional routine experimentation (e.g., peptide mutation and binding analyses) can then be carried out to confirm whether the observed lack of binding of the test antibody is in fact due to binding to the same part of the antigen as the reference antibody or if steric blocking (or another phenomenon) is responsible for the lack of observed binding. Experiments of this sort can be performed using ELISA, RIA, surface plasmon resonance (SPR), flow cytometry or any other quantitative or qualitative antibody-binding assay available in the art.

For instance, it has been shown that antibodies V1, V2, V3, S and O cross-compete for the binding to beta-catenin.

It will also be understood by one skilled in the art that antibodies may undergo a variety of posttranslational modifications. The type and extent of these modifications often depends on the host cell line used to express the antibody as well as the culture conditions. Such modifications may include variations in glycosylation, methionine oxidation, diketopiperazine formation, aspartate isomerization and asparagine deamidation. A frequent modification is the loss of a carboxy-terminal basic residue (such as lysine or arginine) due to the action of carboxypeptidases. Accordingly, the C-terminal lysine of the antibody heavy chain may be absent. In one embodiment, a C-terminal amino acid from the antibody is cleaved during post-translation modifications. In another embodiment, an N-terminal amino acid from the antibody is cleaved during post-translation modifications. In certain further embodiments, antibody variants having one or more amino acid substitutions, insertions, and/or deletions are provided. Sites of interest for substitutional mutagenesis include the CDRs and framework regions. Amino acid substitutions may be introduced into an antibody of interest and the products screened for a desired activity, e.g., retained/improved antigen binding and/or decreased immunogenicity.

In certain embodiments, amino acid sequence variants of the antibodies described herein are contemplated. For example, it may be desirable to improve the binding affinity and/or other biological properties of the antibody. Amino acid sequence variants of the anti-beta-catenin antibody may be prepared by introducing appropriate modifications into the nucleotide sequence encoding the protein, or by peptide synthesis. Such modifications include, for example, deletions from, and/or insertions into and/or substitutions of residues within the amino acid sequences (such as in one or more CDRs and/or framework sequences in the VH domain) of the anti-beta-catenin antibody. Any combination of deletion, insertion, and substitution can be made to arrive at the final construct, provided that the final construct possesses the desired characteristics.

In certain embodiments of the variant VH sequences provided herein, each CDR (or HVR) either is unaltered, or contains no more than one, two or three amino acid substitutions.

It will be appreciated that one or more amino acid substitutions, additions and/or deletions may be made to the CDRs provided by the present invention without significantly altering the ability of the antibody to bind to beta-catenin and to neutralize beta-catenin activity. The effect of any amino acid substitutions, additions and/or deletions can be readily tested by one skilled in the art, for example by using the methods described herein, particularly those illustrated in the Examples, to determine beta-catenin binding and inhibition of the beta-catenin interactions with its natural interacting partners (such as Pontin52, Bcl-9 and lymphoid-enhancing factor-1 (LEF-1)/T-cell factor (TCF) family).

Consequently, in certain embodiments of the variant VH sequences, each CDR either contains no more than one, two or three amino acid substitutions, wherein such amino-acid substitutions are conservative, and wherein the antibody retains its binding properties to beta-catenin. Therefore, herein provided is an antibody that specifically binds to human beta-catenin, wherein said antibody comprises a heavy chain variable region comprising CDR-H1, CDR-H2 and CDR-H3 sequences that have at least 70%, 80%, 90%, 91%, 92%, 93%, 94%, 95%, 96%, 97%, 98%, or 99% identity or similarity to any of the sequences listed in any one of the following i. to iii.: i) a CDR-H1 of SEQ ID NO: 1, 35 or 36; a CDR-H2 of SEQ ID NO: 2, 37 or 38; and a CDR-H3 of SEQ ID NO: 3, 39, 40, 41, 42, 43, 44 or 45; ii) a CDR-H1 of SEQ ID NO: 4; a CDR-H2 of SEQ ID NO: 5 and a CDR-H3 of SEQ ID NO: 6 or 46; iii) a CDR-H1 of SEQ ID NO: 7; a CDR-H2 of SEQ ID NO: 8 and a CDR-H3 of SEQ ID NO: 9, 47, 48, 49 or 50.

Also provided is an antibody that further comprises framework regions (FRs) comprising or consisting of FR1, FR2, FR3 and FR4 sequences that have at least 70%, 80%, 90%, 91%, 92%, 93%, 94%, 95%, 96%, 97%, 98%, or 99% identity or similarity to any of the sequences listed in any one of the following i. to iv.: i. a FR1 comprising SEQ ID NO: 10, 15, or 18; ii. a FR2 comprising SEQ ID NO: 11, 16 or 19; iii. a FR3 comprising SEQ ID NO: 12, 13, 17 or 20; and iv. a FR4 comprising SEQ ID NO: 14.

In an alternative embodiment, an anti-beta-catenin antibody of the present invention comprises a heavy chain variable region comprising a sequence having at least 70%, 80%, 90%, 91%, 92%, 93%, 94%, 95%, 96%, 97%, 98%, or 99% identity or similarity to the sequence given in any one of SEQ ID NO: 21-25, 28, 31 or 33. In a further alternative embodiment, an anti-beta-catenin antibody of the present invention comprises a heavy chain comprising a sequence having at least 70%, 80%, 90%, 91%, 92%, 93%, 94%, 95%, 96%, 97%, 98%, or 99% identity or similarity to the sequence given in any one of SEQ ID NO: 59, 60, 61 or 62.

The beta-catenin antibody variants provided herein by the invention retain the advantageous properties of the parental antibody (i.e. unmodified antibody), i.e. the functional properties herein described. In one example, an anti-beta-catenin antibody variant provided by the invention has a dissociation constant (K_{D}) of 20 nM or less than 20 nM, in particular 15 nM or less than 15 nM, in particular 10 nM or less than 10 nM, such as 9 nM or less than 9 nM, 8 nM or less than 8 nM, 7 nM or less than 7 nM. In certain embodiments, the kD was as low as 6 nM or below, 5 nM or below or even 4 nM or below. The KD can be measured/determined by any standard methods. For instance, the constant of dissociation can be determined by SPR at a temperature of 25° C., between an antibody of the invention and beta-catenin.

Degrees of identity and similarity between sequences can be readily calculated. The “% sequence identity” (or “% sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) amino-acids (e.g., identical amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to obtain the % sequence identity or percent sequence similarity. Methods of alignment of sequences for comparison are well-known in the art. Preferred examples of algorithms that are suitable for determining percent sequence identity and sequence similarity include the BLAST and BLAST 2.0 algorithms. Polypeptide sequences also can be compared using FASTA using default or recommended parameters. FASTA (e.g., FASTA2 and FASTA3) provides alignments and percent sequence identity of the regions of the best overlap between the query and search sequences.

In certain embodiments, substitutions, insertions, or deletions may occur within one or more CDR so long as such alterations do not substantially reduce the ability of the antibody to bind the target. For example, conservative alterations that do not substantially reduce binding affinity may be made in CDRs. Such alterations may be made outside of antigen contacting residues in the CDRs. Conservative substitutions are shown in Table 1 together with more substantial “exemplary substitutions”.

TABLE 1
Examples of amino-acid substitutions

| Original | Exemplary | Conservative |
|---|---|---|
| Residue | Substitutions | Substitution |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys(C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe | Leu |
| Leu (L) | Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; | Leu |

Substantial modifications in the biological properties of an antibody variant can be accomplished by selecting substitutions that differ significantly in their effect on maintaining the structure of the polypeptide backbone in the area of the substitution, the charge or hydrophobicity of the molecule at the target site, or the bulk of the side chain.

One type of substitutional variant involves substituting one or more CDR region residues of a parent antibody (humanized or human antibody). Generally, the resulting variant(s) selected for further study will have changes in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques. Briefly, one or more CDR residues are mutated and the variant antibodies displayed on phage and screened for a particular biological activity (e.g. binding affinity).

Alterations (e.g., substitutions) may be made in CDRs, e.g., to improve antibody affinity. Such alterations may be made in Hyper Variable Region (HVR) “hotspots,” i.e., residues encoded by codons that undergo mutation at high frequency during the somatic maturation process, and/or residues that contact antigen, with the resulting variant VH being tested for binding affinity. Affinity maturation by constructing and reselecting from secondary libraries has been well described in the literature. In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. One of the methods that can be used for identification of residues or regions of an antibody that may be targeted for mutagenesis is alanine scanning mutagenesis. Alternatively, or additionally, an X-ray structure of an antigen-antibody complex can be used to identify contact points between the antibody and its antigen. Variants may be screened to determine whether they contain the desired properties.

Antibodies generated against beta-catenin may be obtained after immunization of an animal by administering beta-catenin or a portion thereof to an animal, preferably a non-human animal, using well-known and routine protocols. Many animals, such as rabbits, mice, rats, sheep, cows, llamas, camels or pigs may be immunized.

Monoclonal antibodies may be made by a variety of techniques, including but not limited to, the hybridoma method, recombinant DNA methods, phage-display methods, and methods utilizing transgenic animals containing all or a part of the human immunoglobulin loci. One non-limiting method for making antibodies is described in the example section.

Herein described is also a method of identifying an antibody according to the invention, said method comprising:
a) immunizing a non-human animal with a beta-catenin immunogenic composition;
b) recovering B cells from said non-human mammal;
c) selecting the antibodies produced by said B cells that have at least one or more of the following properties:
   i. bind to beta-catenin with an affinity represented by a dissociation constant K_{D} of less than 20 nM;
   ii. block the binding of any one or more of Pontin52, Bcl-9 and TCF to beta-catenin; and/or
   iii. neutralise or inhibit the transcriptional activating activity of beta-catenin.

The present invention also provides an isolated polynucleotide encoding the antibodies according to the present invention.

The isolated polynucleotide according to the present invention may comprise synthetic DNA, for instance produced by chemical processing, cDNA, genomic DNA or yet mRNA or any combination thereof.

The present invention also provides for a cloning or expression vector comprising one or more polynucleotides described herein. In one example, the cloning or expression vector according to the present invention comprises one or more isolated polynucleotides as described above. Standard techniques of molecular biology may be used to prepare DNA sequences coding for the antibody of the present invention. Desired DNA sequences may be synthesized completely or in part using oligonucleotide synthesis techniques. Site-directed mutagenesis and polymerase chain reaction (PCR) techniques may be used as appropriate.

General methods by which the vectors may be constructed, transfection methods and culture methods are well known to those skilled in the art.

Also provided is a host cell comprising one or more isolated polynucleotide sequences according to the invention or one or more cloning or expression vectors comprising one or more isolated polynucleotide sequences encoding an antibody of the present invention. Any suitable host cell/vector system may be used for expression of the polynucleotide sequences encoding the antibody of the present invention. Bacterial, for example E. coli, and other microbial systems may be used. In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. Further host cells that can be used are any eukaryotic cells, for example mammalian. Suitable mammalian host cells include Chinese Hamster Ovary (CHO) cell, human embryonic cell (HEK cell) or lymphoid cell (e.g., Y0, NS0, Sp20 cell). Suitable types of CHO cells according to the present invention may include CHO and CHO-K1 cells including dhfr-CHO cells, such as CHO-DG44 cells and CHO-DXB11 cells and which may be used with a DHFR selectable marker or CHOK1-SV cells which may be used with a glutamine synthetase selectable marker. Other cell types of use in expressing antibodies include lymphocytic cell lines, e.g., NS0 myeloma cells and SP2 cells, COS cells; or human embryonic cells such as HEK293, HEK293F, HEK293S or EK293T. The host cell may be stably transformed or transfected with the isolated polynucleotide sequences or the expression vectors according to the present invention.

The present invention also provides a process for the production of an antibody according to the present invention comprising culturing a host cell according to the present invention under conditions suitable for producing the antibody according to the invention and isolating the antibody. The present invention also provides a process for the production of a pharmaceutical composition comprising an antibody according to the present invention comprising culturing a host cell according to the present invention under conditions suitable for producing the antibody according to the invention, isolating the antibody, and formulating the antibody into a pharmaceutical composition.

The antibody may comprise only a heavy chain polypeptide, in which case only a heavy chain polypeptide coding sequence needs to be used to transfect the host cells (in only one vector). For production of antibodies comprising both heavy and light chains, the cell line may be transfected with two vectors, a first vector encoding a light chain polypeptide and a second vector encoding a heavy chain polypeptide. Alternatively, a single vector may be used, the vector including sequences encoding light chain and heavy chain polypeptides.

Thus, here is provided a process for culturing a host cell and expressing an antibody according to the invention, isolating the antibody and optionally purifying said antibody to provide an isolated antibody.

The present invention also provides a process for the production of an antibody according to the present invention comprising culturing a host cell containing a vector of the present invention under conditions suitable for leading to expression of protein from DNA encoding the antibody molecule of the present invention and isolating the antibody molecule.

In one embodiment there is provided a purified antibody, for example a humanized antibody, in particular an antibody according to the invention, in substantially purified form, in particular free or substantially free of endotoxin and/or host cell protein or DNA.

The antibody according to the invention may be provided in a pharmaceutical composition. Therefore, the present invention also provides for a pharmaceutical comprising the antibody according to the present invention, or a polynucleotide encoding the antibody according to the present invention, in combination with one or more of a pharmaceutically acceptable carriers, excipients and/or diluents. Preferably, the pharmaceutical composition comprises an antibody which specifically binds beta-catenin, or a polynucleotide encoding such antibody, and one or more pharmaceutically acceptable carriers, excipients and/or diluents, wherein said antibody comprises a heavy chain variable region comprising:
i. a CDR-H1 according to SEQ ID NO: 1, 35 or 36; a CDR-H2 according to SEQ ID NO: 2, 37 or 38; and a CDR-H3 according to SEQ ID NO: 3, 39, 40, 41, 42, 43, 44 or 45;
ii. a CDR-H1 according to SEQ ID NO: 4; a CDR-H2 according to SEQ ID NO: 5 and a CDR-H3 according to SEQ ID NO: 6 or 46;
iii. a CDR-H1 according to SEQ ID NO: 7; a CDR-H2 according to SEQ ID NO: 8 and a CDR-H3 according to SEQ ID NO: 9, 47, 48, 49 or 50; or
iv. a sequence according to any one of SEQ ID NO: 21-25, 28, 31 or 33;
v. a sequence according to any one of SEQ ID NO: 59, 60, 61 or 62;
vi. CDR-H1, CDR-H2 and CDR-H3 that have at least 70%, 80%, 90%, 91%, 92%, 93%, 94%, 95%, 96%, 97%, 98%, or 99% identity or similarity to any of the sequences listed in any one of i. to iii.; or
vii. a sequence that has at least 70%, 80%, 90%, 91%, 92%, 93%, 94%, 95%, 96%, 97%, 98%, or 99% identity or similarity to any of the sequences listed in any one of iv. to v.

The pharmaceutical compositions according to the invention can be used in therapy. Said pharmaceutical composition may be administered suitably to a patient to identify the therapeutically effective amount required. For any antibody, the therapeutically effective amount can be estimated initially either in cell culture assays or in animal models, usually in rodents, rabbits, dogs, pigs or primates. The animal model may also be used to determine the appropriate concentration range and route of administration. Such information can then be used to determine useful doses and routes for administration in humans.

The precise therapeutically effective amount for a human subject will depend upon the severity of the disease state, the general health of the subject, the age, weight and gender of the subject, diet, time and frequency of administration, drug combination(s), reaction sensitivities and tolerance/response to therapy. Generally, a therapeutically effective amount will be from 0.01 mg/kg to 500 mg/kg, for example 0.1 mg/kg to 200 mg/kg, such as 100 mg/kg. Pharmaceutical compositions may be conveniently presented in unit dose forms containing a predetermined amount of an active agent of the invention per dose.

The pharmaceutical compositions according to the invention preferably comprise not only the antibody according to the invention, or a polynucleotide encoding such antibody, but also one or more pharmaceutically acceptable carriers, excipients (such as buffer, stabilizer, suspending, preservative, and/or dispersing agents or other materials well known to those skilled in the art) and/or diluent. Such materials should be non-toxic and should not interfere with the efficacy of the active ingredient. Examples of the carriers, excipients and/or diluents, and method for preparing pharmaceutical compositions can be found in Remington's Pharmaceutical Sciences, 20th Edition, 2000, pub. Lippincott, Williams & Wilkins.

Suitable forms for administration include forms suitable for parenteral administration, e.g. by injection or infusion, for example by bolus injection or continuous infusion, in intravenous, inhalable or sub-cutaneous form. Where the product is for injection or infusion, it may take the form of a suspension, solution or emulsion in an oily or aqueous vehicle. Alternatively, the antibody according to the invention may be in dry form, for reconstitution before use with an appropriate sterile liquid. Solid forms suitable for solution in, or suspension in, liquid vehicles prior to injection may also be prepared.

As the antibodies according to the invention are intrabodies, to increase their availability intracellularly, alternative delivery methods can be used such as their attachment to cell penetrating peptides such as TAT (van den Berg and Dowdy, 2011), manipulation of the intrabody pI to create cell penetrating antibodies (transbodies) (Lafaye et al., 2011), delivery using viral nanoparticles (Yildiz et al., 2011) or yet delivery by carbon nanotubes (Shi Kam et al., 2004). Once formulated, the compositions of the invention can be administered directly to the subject or can be administered after reconstitution depending on the form.

Accordingly, provided herein is the use of an antibody according to the invention, or a polynucleotide encoding such antibody, for the manufacture of a medicament.

Preferably, the pharmaceutical composition according to the present invention is adapted for administration to primate, such as human or non-human subjects.

Also herein encompassed is a pharmaceutical comprising an antibody that specifically binds beta-catenin, or a polynucleotide encoding such antibody, and one or more pharmaceutically acceptable carriers, excipients and/or diluents, for use in therapy, wherein said antibody comprises a heavy chain variable region comprising:
i. a CDR-H1 of SEQ ID NO: 1, 35 or 36; a CDR-H2 of SEQ ID NO: 2, 37 or 38; and a CDR-H3 of SEQ ID NO: 3, 39, 40, 41, 42, 43, 44 or 45;
ii. a CDR-H1 of SEQ ID NO: 4; a CDR-H2 of SEQ ID NO: 5 and a CDR-H3 of SEQ ID NO: 6 or 46;
iii. a CDR-H1 of SEQ ID NO: 7; a CDR-H2 of SEQ ID NO: 8 and a CDR-H3 of SEQ ID NO: 9, 47, 48, 49 or 50;
iv. a sequence according to any one of SEQ ID NO: 21-25, 28, 31 or 33;
V. a sequence according to any one of SEQ ID NO: 59, 60, 61 or 62;
vi. CDR-H1, CDR-H2 and CDR-H3 that have at least 70%, 80%, 90%, 91%, 92%, 93%, 94%, 95%, 96%, 97%, 98%, or 99% identity or similarity to any of the sequences listed in any one of i. to iii.; or
vii. a sequence that has at least 70%, 80%, 90%, 91%, 92%, 93%, 94%, 95%, 96%, 97%, 98%, or 99% identity or similarity to any of the sequences listed in any one of iv. to v.

Herein also encompassed is an antibody according to the invention, a polynucleotide according to the invention or a pharmaceutical composition according to the invention for use in therapy, in particular for use in the treatment of a disorder or condition as described herein. Such antibody is administered in a therapeutically effective amount.

The present invention provides a method of treating a disorder or condition as described herein in a subject in need thereof, the method comprising administering to the subject an antibody, a polynucleotide or a pharmaceutical composition according to the present invention. Such antibody is administered in a therapeutically effective amount.

The present invention also provides the use of an antibody, a polynucleotide or a pharmaceutical composition of the invention for the manufacture of a medicament, in particular for use in the treatment of a disorder or condition as described herein.

In one embodiment, the disorder or condition in the context of the present invention as a whole is characterized by over expression of beta-catenin, enhanced abundance of beta-catenin and/or increased transcriptional activity of beta-catenin. Preferably, the disorder or condition is a cancer, such as (but not limited to) colorectal cancers, adenomatous polyposis (FAP), colon cancer, melanoma, hepatocellular carcinoma, ovarian cancer, endometrial cancer, medulloblastoma pilomatricomas, and prostate cancer. Alternatively, disorder or condition in the context of the present invention as a whole can be a neurodegenerative disorder, such as Huntington's disease, Alzheimer's disease, Parkinson's disease, prion diseases, multiple sclerosis, amyotrophic lateral sclerosis or schizophrenia, or yet a disorder such as bone density disorders, schizophrenia, type II diabetes, rheumatoid arthritis, oligodentonia, osteoporosis-pseudoglioma syndrome; familial exudative vitreoretinopathy or yet vascular calcification.

In an alternative embodiment, herein also provided is an antibody according to the invention, a polynucleotide according to the invention or a pharmaceutical composition according to the invention for use in preventing the excess growth of polyps in FAP patients.

### BRIEF DESCRIPTION OF THE DRAWINGS

FIG. 1: Screening for intracellular function of VHH intrabodies transiently transfected into HEK293 bioassay cells. Wnt signalling was induced by co-transfection of the PPSP gene. A)C)E)G)I): Wnt inducible Firefly luciferase activity, results plotted as percentage of control VHH (CAb) transfected cells. B)D)F)H)J): Constitutive Renilla luciferase activity, results plotted as percentage of control VHH (CAb) transfected cells. The star symbol denotes a VHH intrabody of interest. Each set of graphs represents a different plate. Error bars show SEM.

FIG. 2: Secondary screen for intracellular function of VHH intrabodies transiently transfected into HEK293 bioassay cells. Wnt signalling was induced by co-transfection of the Wnt1 gene. A) C) Firefly luciferase activity, results are plotted as fold stimulation over vector transfected inactivated cells, all conditions performed in triplicate. B)D) Renilla luciferase activity, results are plotted as raw signals. The star symbol denotes previously selected intrabody of interest. Error bars show SEM.

FIG. 3: Sequence alignments of function modifying intrabodies VHH sequences which showed functional activity against beta-catenin transcriptional activity in HEK293 luciferase-based bioassay. The three CDRs are highlighted in bold and underlined fonts.

FIG. 4: Alignments of parent VHHs and designed CDR3 mutants.

FIG. 5: Reducing anti-beta-catenin Western blots probed by the intrabodies. Gel A:) IBV1-scFc (23 nM), Gel B: IBV2-scFc (110 nM), Gel C: IBV3-scFc (85 nM) and Gel D: IBV1.CDRAAA-scFc (16 nM) transient supernatants. Any binding was detected with t anti-mouse Fc HRP antibody. In each gel, Lane 1) 15 ng beta-catenin, Lane 2) 7.5 ng beta-catenin, Lane 3) 3.75 ng beta-catenin.

FIG. 6: beta-catenin binding ELISA results for parent VHHs and CDR3 mutants. Biotinylated beta-catenin was captured on to streptavidin coated plates, and VHHscFc constructs were then added in half log dilutions from 32 nM and revealed with anti-mouse Fc HRP. The plates were read at 630 and 490 nm and ΔOD recorded. The negative control is a ‘no DNA’ control transfection culture supernatant.

FIG. 7: Activity of parents intrabodies and CDR3 mutant control intrabodies in bioassay. Parent intrabody and selected CDR3 mutant control intrabody pairs of IBV1 with IBV1.CDRAAA, IBV2 with IBV2.CDRAAA and IBV3 with IBV3.CDRAAA (1) were tested for firefly and Renilla luciferase activity in the bioassay. “A” and “B”: Firefly luciferase activity. Results were plotted as fold stimulation over transfected but inactivated cells. All conditions were performed in five replicates and are representative of four independent experiments. “C” and “D”: Renilla luciferase activity. Results were plotted as raw data. **** denotes p≤0.0001, *** denotes p of 0.0001-0.001, ** denotes p of 0.001-0.01, * denotes p of 0.01-0.05 for a two-tailed unpaired Student's t-test. Error bars show SEM

Description of the sequences:

| SEQ | | |
|---|---|---|
| ID NO | Sequence | Name |
| 1 | GXISSNNVMG | CDR-H1 |
| | With X = any amino acid or alternatively | |
| | X = serine or threonine | |
| 2 | SXTDSDLANYAASVKG | CDR-H2 |
| | With X = any amino acid or alternatively | |
| | X = isoleucine or valine | |
| 3 | TX₁MTYPPX₂IS | CDR-H3 |
| | With X₁ and/or X₂ = any amino acid, or | |
| | alternatively X₁ = isoleucine or methionine | |
| | and/or X₂ = threonine or isoleucine | |
| 4 | GVSFSRTSLG | CDR-H1 |
| 5 | VISWIGGTTYYADSLKG | CDR-H2 |
| 6 | SNGVLPRTEGAFAS | CDR-H3 |
| 7 | EGTFRTNVMG | CDR-H1 |
| 8 | AISWSGGITHYSNSVKG | CDR-H2 |
| 9 | DPSTTPWNLX₁X₂X₃VNEYEY | CDR-H3 |
| | With X₁, X₂ and/or X₃ = any amino acid, or | |
| | alternatively X₁ and/or X₃ = arginine | |
| | or alanine and/or X₂ = valine or alanine | |
| 10 | QVQLVXSGGGLVQPGGSLRLSCAAS | FR-1 |
| | With X = any amino acid or alternatively | |
| | X = Glutamine or Glutamic Acid | |
| 11 | WYRQGPGKQREFVA | FR-2 |
| 12 | RFTISRDNAKNTVNLQMNSLRPEDTXVYYCRL | FR-3 |
| | With X = any amino acid or alternatively | |
| | X = Aspartic Acid or Glycine | |
| 13 | RFTISRDNAKNTVNLQMNSLRPEDTXVYYCNA | FR-3 |
| | With X = any amino acid or alternatively | |
| | X = Aspartic Acid or Glycine | |
| 14 | WGQGTQVTVSS | FR-4 |
| 15 | QVQLVXSGGGLVQAGGSLRLSCAAS | FR-1 |
| | With X = any amino acid or alternatively | |
| | X = Glutamine or Glutamic Acid | |
| 16 | WFRQAPGKEREFVS | FR-2 |
| 17 | RFTISRDNAKSTVYLQMNSLKPEDTAX₁YYCAX₂ | FR-3 |
| | With X₁ and/or X₂ = any amino acid, or | |
| | alternatively X₁ = leucine or valine | |
| | and/or X₂ = alanine or isoleucine | |
| 18 | QVQLVQSGGGLVQAGDSLRLACAAS | FR-1 |
| 19 | WFRQAPGKEREFVA | FR-2 |
| 20 | RFAISRDNAKNTVYLQMNSLKPEDTAVYYCAA | FR-3 |
| 21 | QVQLVQSGGGLVQPGGSLRLSCAASGSISSNNVMGWYRQGPGKQREFV | VH-V1 |
| | ASITDSDLANYAASVKGRFTISRDNAKNTVNLQMNSLRPEDTDVYYCRLT | |
| | MMTYPPTISWGQGTQVTVSS | |
| 22 | QVQLVQSGGGLVQPGGSLRLSCAASGSISSNNVMGWYRQGPGKQREFV | VH-V2 |
| | ASITDSDLANYAASVKGRFTISRDNAKNTVNLQMNSLRPEDTDVYYCRLT | |
| | MMTYPPIISWGQGTQVTVSS | |
| 23 | QVQLVESGGGLVQPGGSLRLSCAASGTISSNNVMGWYRQGPGKQREFV | VH-V3 |
| | ASVTDSDLANYAASVKGRFTISRDNAKNTVNLQMNSLRPEDTGVYYCRLT | |
| | IMTYPPTISWGQGTQVTVSS | |
| 24 | QVQLVQSGGGLVQPGGSLRLSCAASGSISSNNVMGWYRQGPGKQREFV | VH-V1. |
| | ASITDSDLANYAASVKGRFTISRDNAKNTVNLQMNSLRPEDTDVYYCRLT | CDRA |
| | MMTAPPTISWGQGTQVTVSS | |
| 25 | QVQLVQSGGGLVQPGGSLRLSCAASGSISSNNVMGWYRQGPGKQREFV | VH-V1. |
| | ASITDSDLANYAASVKGRFTISRDNAKNTVNLQMNSLRPEDTDVYYCRLT | CDRAA |
| | MMTYAATISWGQGTQVTVSS | |
| 26 | QVQLVQSGGGLVQPGGSLRLSCAASGSISSNNVMGWYRQGPGKQREFV | VH-V1. |
| | ASITDSDLANYAASVKGRFTISRDNAKNTVNLQMNSLRPEDTDVYYCRLT | CDRAAA |
| | MMTAAATISWGQGTQVTVSS | |
| 27 | QVQLVQSGGGLVQPGGSLRLSCAASGSISSNNVMGWYRQGPGKQREFV | VH-V1. |
| | ASITDSDLANYAASVKGRFTISRDNAKNTVNLQMNSLRPEDTDVYYCNAN | CDRgraft |
| | LRPRRWSAWQNYWGQGTQVTVSS | |
| 28 | QVQLVESGGGLVQAGGSLRLSCAASGVSFSRTSLGWFRQAPGKEREFV | VH-S |
| | SVISWIGGTTYYADSLKGRFTISRDNAKSTVYLQMNSLKPEDTALYYCAIS | |
| | NGVLPRTEGAFASWGQGTQVTVSS | |
| 29 | QVQLVQSGGGLVQAGGSLRLSCAASGVSFSRTSLGWFRQAPGKEREFV | VH-S. |
| | SVISWIGGTTYYADSLKGRFTISRDNAKSTVYLQMNSLKPEDTALYYCAIS | CDRAAA |
| | NGVLPATAGAAASWGQGTQVTVSS | |
| 30 | QVQLVQSGGGLVQAGGSLRLSCAASGVSFSRTSLGWFRQAPGKEREFV | VH-S. |
| | SVISWIGGTTYYADSLKGRFTISRDNAKSTVYLQMNSLKPEDTAVYYCAAR | CDRgraft |
| | PAEQYGSLRPYNYWGQGTQVTVSS | |
| 31 | QVQLVQSGGGLVQAGDSLRLACAASEGTFRTNVMGWFRQAPGKEREFV | VH-O |
| | AAISWSGGITHYSNSVKGRFAISRDNAKNTVYLQMNSLKPEDTAVYYCAA | |
| | DPSTTPWNLRVRVNEYEYWGQGTQVTVSS | |
| 32 | QVQLVQSGGGLVQAGDSLRLACAASEGTFRTNVMGWFRQAPGKEREFV | VH-O. |
| | AAISWSGGITHYSNSVKGRFAISRDNAKNTVYLQMNSLKPEDTAVYYCAA | CDRAAA(1) |
| | DPSTTAAALRVRVNEYEYWGQGTQVTVSS | |
| 33 | QVQLVQSGGGLVQAGDSLRLACAASEGTFRTNVMGWFRQAPGKEREFV | VH-O. |
| | AAISWSGGITHYSNSVKGRFAISRDNAKNTVYLQMNSLKPEDTAVYYCAA | CDRAAA(2) |
| | DPSTTPWNLAAAVNEYEYWGQGTQVTVSS | |
| 34 | QVQLVQSGGGLVQAGDSLRLACAASEGTFRTNVMGWFRQAPGKEREFV | VH-O.graft |
| | AAISWSGGITHYSNSVKGRFAISRDNAKNTVYLQMNSLKPEDTAVYYCAA | |
| | DRRVGATPNSYFDAKFYDPWGQGTQVTVSS | |
| 35 | GSISSNNVMG | H-CDR1 |
| 36 | GTISSNNVMG | H-CDR1 |
| 37 | SITDSDLANYAASVKG | H-CDR2 |
| 38 | SVTDSDLANYAASVKG | H-CDR2 |
| 39 | TMMTYPPTIS | H-CDR3 |
| 40 | TMMTYPPIIS | H-CDR3 |
| 41 | TIMTYPPTIS | H-CDR3 |
| 42 | TMMTAPPTIS | H-CDR3 |
| 43 | TMMTYAATIS | H-CDR3 |
| 44 | TMMTAAATIS | H-CDR3 |
| 45 | NLRPRRWSAWQNY | H-CDR3 |
| 46 | RPAEQYGSLRPYNY | H-CDR3 |
| 47 | DPSTTPWNLRVRVNEYEY | H-CDR3 |
| 48 | DPSTTAAALRVRVNEYEY | H-CDR3 |
| 49 | DPSTTPWNLAAAVNEYEY | H-CDR3 |
| 50 | DRRVGATPNSYFDAKYDP | H-CDR3 |
| 51 | MATQADLMELDMAMEPDRKAAVSHWQQQSYLDSGIHSGATTTAPSLSG | beta- |
| | KGNPEEEDVDTSQVLYEWEQGFSQSFTQEQVADIDGQYAMTRAQRVRA | catenin |
| | AMFPETLDEGMQIPSTQFDAAHPTNVQRLAEPSQMLKHAVVNLINYQDDA | |
| | ELATRAIPELTKLLNDEDQVVVNKAAVMVHQLSKKEASRHAIMRSPQMVS | |
| | AIVRTMQNTNDVETARCTAGTLHNLSHHREGLLAIFKSGGIPALVKMLGSP | |
| | VDSVLFYAITTLHNLLLHQEGAKMAVRLAGGLQKMVALLNKTNVKFLAITT | |
| | DCLQILAYGNQESKLIILASGGPQALVNIMRTYTYEKLLWTTSRVLKVLSVC | |
| | SSNKPAIVEAGGMQALGLHLTDPSQRLVQNCLWTLRNLSDAATKQEGME | |
| | GLLGTLVQLLGSDDINVVTCAAGILSNLTCNNYKNKMMVCQVGGIEALVRT | |
| | VLRAGDREDITEPAICALRHLTSRHQEAEMAQNAVRLHYGLPVVVKLLHP | |
| | PSHWPLIKATVGLIRNLALCPANHAPLREQGAIPRLVQLLVRAHQDTQRRT | |
| | SMGGTQQQFVEGVRMEEIVEGCTGALHILARDVHNRIVIRGLNTIPLFVQL | |
| | LYSPIENIQRVAAGVLCELAQDKEAAEAIEAEGATAPLTELLHSRNEGVATY | |
| | AAAVLFRMSEDKPQDYKKRLSVELTSSLFRTEPMAWNETADLGLDIGAQG | |
| | EPLGYRQDDPSYRSFHSGGYGQDALGMDPMMEHEMGGHHPGADYPVD | |
| | GLPDLGHAQDLMDGLPPGDSNQLAWFDTDL | |
| 52 | SAGGTGCAGCTGGTRGAGTCTGGGGGAG | Primer |
| 53 | GGTACGTGCTGTTGAACTGTTCC | Primer |
| 54 | GGSSTASGSGSGGSGTAGSSGGAGSSGGSTTAGGSASGSGSTGSGTG | linker |
| | GASSGGASGASG | |
| 55 | APELLGGPSVFIFPPKPKDVLSISGRPEVTCVVVDVGQEDPEVSFNWYIDG | Exemplary |
| | AEVRTANTRPKEEQFNSTYRVVSVLPIQHQDWLTGKEFKCKVNNKALPAP | CH2/CH3 |
| | IEKTISKAKGQTREPQVYTLAPHREELAKDTVSVTCLVKGFYPPDINVEWQ | domains |
| | RNRQPEPEGTYATTPPQLDNDGTYFLYSKLSVGKNTWQRGETFTCVVMH | |
| | EALHNHYTQKSITQSS | |
| 56 | GPELLGGPTVFIFPPKPKDVLSITRKPEVTCVVVDVGKEDPEIEFSWSVDD | Exemplary |
| | TEVHTAETKPKEEQFNSTYRVVSVLPIQHQDWLTGKEFKCKVNNKALPAP | CH2/CH3 |
| | IERTISKAKGQTREPQVYTLAPHREELAKDTVSVTCLVKGFFPADINVEWQ | domains |
| | RNGQPESEGTYATTPPQLDNDGTYFLYSKLSVGKNTWQQGEVFTCVVM | |
| | HEALHNHSTQKSISQSP | |
| 57 | AHHSEDPSSKCPKCP | Exemplary |
| | | hinge |
| 58 | EPKTPKPQPQPQPQPQPNPTTESKCPKCP | Exemplary |
| | | hinge |
| 59 | QVQLVQSGGGLVQPGGSLRLSCAASGSISSNNVMGWYRQGPGKQREFV | Exemplary |
| | ASITDSDLANYAASVKGRFTISRDNAKNTVNLQMNSLRPEDTDVYYCRLT | HCab w/o |
| | MMTYPPTISWGQGTQVTVSSAPELLGGPSVFIFPPKPKDVLSISGRPEVT | linker |
| | CVVVDVGQEDPEVSFNWYIDGAEVRTANTRPKEEQFNSTYRVVSVLPIQH | |
| | QDWLTGKEFKCKVNNKALPAPIEKTISKAKGQTREPQVYTLAPHREELAK | |
| | DTVSVTCLVKGFYPPDINVEWQRNRQPEPEGTYATTPPQLDNDGTYFLY | |
| | SKLSVGKNTWQRGETFTCVVMHEALHNHYTQKSITQSS | |
| 60 | QVQLVQSGGGLVQAGDSLRLACAASEGTFRTNVMGWFRQAPGKEREFV | Exemplary |
| | AAISWSGGITHYSNSVKGRFAISRDNAKNTVYLQMNSLKPEDTAVYYCAA | HCab w/o |
| | DPSTTPWNLRVRVNEYEYWGQGTQVTVSSGPELLGGPTVFIFPPKPKDV | linker |
| | LSITRKPEVTCVVVDVGKEDPEIEFSWSVDDTEVHTAETKPKEEQFNSTY | |
| | RVVSVLPIQHQDWLTGKEFKCKVNNKALPAPIERTISKAKGQTREPQVYTL | |
| | APHREELAKDTVSVTCLVKGFFPADINVEWQRNGQPESEGTYATTPPQL | |
| | DNDGTYFLYSKLSVGKNTWQQGEVFTCVVMHEALHNHSTQKSISQSP | |
| 61 | QVQLVQSGGGLVQPGGSLRLSCAASGSISSNNVMGWYRQGPGKQREFV | Exemplary |
| | ASITDSDLANYAASVKGRFTISRDNAKNTVNLQMNSLRPEDTDVYYCRLT | HCab w |
| | MMTYPPTISWGQGTQVTVSSAHHSEDPSSKCPKCPAPELLGGPSVFIFPP | linker |
| | KPKDVLSISGRPEVTCVVVDVGQEDPEVSFNWYIDGAEVRTANTRPKEEQ | |
| | FNSTYRVVSVLPIQHQDWLTGKEFKCKVNNKALPAPIEKTISKAKGQTREP | |
| | QVYTLAPHREELAKDTVSVTCLVKGFYPPDINVEWQRNRQPEPEGTYATT | |
| | PPQLDNDGTYFLYSKLSVGKNTWQRGETFTCVVMHEALHNHYTQKSITQ | |
| | SS | |
| 62 | QVQLVQSGGGLVQAGDSLRLACAASEGTFRTNVMGWFRQAPGKEREFV | Exemplary |
| | AAISWSGGITHYSNSVKGRFAISRDNAKNTVYLQMNSLKPEDTAVYYCAA | HCab w |
| | DPSTTPWNLRVRVNEYEYWGQGTQVTVSSEPKTPKPQPQPQPQPQPNP | linker |
| | TTESKCPKCPGPELLGGPTVFIFPPKPKDVLSITRKPEVTCVVVDVGKEDP | |
| | EIEFSWSVDDTEVHTAETKPKEEQFNSTYRVVSVLPIQHQDWLTGKEFKC | |
| | KVNNKALPAPIERTISKAKGQTREPQVYTLAPHREELAKDTVSVTCLVKGF | |
| | FPADINVEWQRNGQPESEGTYATTPPQLDNDGTYFLYSKLSVGKNTWQQ | |
| | GEVFTCVVMHEALHNHSTQKSISQSP | |

Note:
in the above Table, “VH” (in the right column) sands for variable heavy chain.

### EXAMPLES

### Methods

Production of recombinant beta-catenin protein: The full-length human beta-catenin cDNA clone was obtained from Origene. Beta-catenin was expressed and purified essentially as described in the literature (Xing Y et al., 2008).

Generation of immune VHH phage library: Two male llamas were immunised over 3 months with 5 doses of 0.25 mg recombinant beta-catenin in Freund's adjuvant. PBMCs were collected from these animals at different point in time (bleedings) and RNA purified using RNeasy kit (Qiagen). VH genes were amplified using primers ‘llimmlibfor’ (SEQ ID No.52) and ‘CH2Ig primer’ 54 (SEQ ID No.53) by PCR following RT of purified RNA using oligodT primers. VHH genes were specifically distinguished from the resulting PCR product based on size, re-amplified using specific primers and cloned into pASTT. The ligated product was electroporated into XL-1 Blue E. coli (Stratagene), and the library was estimated to contain 2×10⁸ members. Phage particles were prepared using standard methods.

Phage panning: In the first panning experiment, the immune library was incubated in beta-catenin-conjugated immunotubes blocked in 3% bovine serum albumin (BSA). Two separate schemes of washing were then performed: a low wash protocol (5 washes in round 1 and 10 washes in round 2) and a high wash protocol (20 washes in round 1 and 40 washes in round 2), prior to elution of phage with 0.1 M HCl, which was subsequently neutralised. In the second round of panning, input phage from both wash strategies were panned against both beta-catenin conjugated and unconjugated immunotubes. One round of panning was also completed on antigen in solution. Recombinant biotinylated beta-catenin was incubated (rolling) with 6×10¹¹ library phage particles, previously blocked in phosphate-buffered saline (PBS) containing 2.5% milk. beta-catenin and associated phage were then captured out of solution using M280 streptavidin-conjugated Dynabeads® (Invitrogen). The beads were washed by magnetic capture 4 times, and bound phages were eluted as above. Small scale phage rescues were performed for individual E. coli colonies picked from output phage infected colonies from each round of all panning conditions. Each sample was tested for binding to beta-catenin or streptavidin by ELISA. A positive ‘hit’ was determined to be any sample with an antigen binding signal greater than 3 times the negative signal.

Bioassay screening: HEK293 cells stably transfected with Tcf-firefly Luciferase reporter construct according to standard protocol and were grown in DMEM (Gibco Life Technologies) supplemented with 10% fetal bovine serum, 2 mM glutamine and non-essential amino acids (Gibco Life Technologies). Cells were seeded at 5×10⁴/well in white poly-D-lysine coated 96-well plates and were transfected with expression plasmids (including 10 ng of Renilla Luciferase pGL4.74 plasmid from Promega) using Lipofectamine 2000 (Gibco Life Technologies) according to the manufacturer's instructions. Plates were incubated for 40 hours at 37° C. in 5% CO2. When required Wnt3a conditioned media or LiCl2 were added, 18 hours post transfection. Following the incubation, firefly and Renilla luciferase activity were measured using Dual-Glo® (Promega) as manufacturer's instructions. Luminescence was recorded using a Biotek Synergy plate reader.

ELISAs: ELISA plates were coated overnight at 4° C. Washes were performed between each step of the assay and consisted of 4 washes in PBS (containing 0.1% Tween20). All plates were blocked in PBS containing 3% BSA, and all samples were blocked in PBS containing 2.5% milk, for at least an hour prior to addition of screening samples to ELISA plates. Following the final wash, TMB (Calbiochem) was added, and the OD of plates was read at 630 and 490 nm, with delta-OD (ΔOD) recorded using a Biotek Synergy plate reader.

Beta-catenin binding ELISA: ELISA plates were coated with streptavidin (2 mg/mL). Biotinylated beta-catenin (1 mg/mL) was added in PBS, for 30 minutes. Blocked samples were added (phage samples or transient VHHscFc samples). After an hour, bound samples were detected using a suitable antibody at 1 in 5,000 dilution in PBS (containing 3% BSA). Detection antibodies were goat anti-M13-HRP (GE Healthcare) or goat anti-mouse Fc HRP (Jackson ImmunoResearch).

Streptavidin binding ELISA: ELISA plates were coated with streptavidin (2 mg/ml). Blocked samples were added. After an hour bound samples were detected using a suitable antibody at 1 in 5,000 dilution in PBS (+3% BSA).

Confocal microscopy: HEK293 cells transfected with Myc-His tagged VHH constructs were attached to poly-D-lysine coated 8 well culture microscope slides in complete media. The cells were fixed in 4% paraformaldehyde and blocked/permeabilised in TBS block (containing 0.3% Triton 100, 1% BSA and 5% goat serum) for 1 hour. Anti-myc (9E10) Alexa Fluor® 488 (Gentaur), and anti beta-catenin (6B3) (Cell Signalling Technologies) were then added and incubated overnight at 4° C. in a moist chamber. The anti-beta-catenin detection antibody, anti-rabbit Alexa Fluor® 647 (Jackson ImmunoResearch), was added at a 1 in 1000 dilution, and incubated for 1 hour in the dark. Finally DAPI ProLong® Gold Antifade (Invitrogen) was added with a coverslip. Images were captured using a Leica TCS SP5 microscope.

VHH-scFc protein production: HEK293 cells were transfected with VHH-scFc expression vectors using 293Fectin™ (Invitrogen), in accordance with the manufacturer's instructions. The single chain mouse IgG Fc constructs comprised VHH, hinge, CH2 and CH3 domains, linked to another hinge, CH2, CH3 sequence through a 59 amino acid linker (SEQ ID NO: 54). This construct had previously been shown to aid extracellular expression, purification and orientated immobilisation, and as a tag for VHHs (data not shown). Transfected HEK293 cells were incubated at 37 C, with 5% CO2 on a shaking platform for 5-6 days. The cells were then harvested from the cultures by centrifugation at 423×g, and the VHH-scFc containing supernatant was stored at 4° C.

Immunoprecipitation: HEK293 bioassay cells, 2×10⁶, were washed in ice-cold PBS, and resuspended in 200 mL of ice-cold lysis buffer (including protease inhibitor cocktail). The samples were then rolled at 4° C. for 30 minutes, followed by centrifugation for 15 minutes at 10,000×g at 4° C. The HEK293 cell lysate supernatant was then removed into a fresh chilled microfuge tube. VHH-scFcs were conjugated to sheep anti-mouse Fc Dynabeads® (Invitrogen) as manufacturer's instructions. The beads were then each added to 500 mL of HEK293 cell lysate and rolled at 4° C. overnight. Each set of beads were magnetically captured out of solution and individually washed 3 times in fresh cell lysis buffer. Protein was then eluted from the beads by boiling in LDS sample buffer including dithiothreitol (DTT) (Invitrogen) for 5 minutes. Samples were then analyzed by anti-beta-catenin and anti-VHH-scFc Western blots.

SDS-PAGE and immunological detection of proteins (Western blotting): Appropriate amounts of protein samples were separated by SDS-PAGE on 4-12% bis-tris gels (Invitrogen), before being electro-blotted using iBLOT (Invitrogen) onto PVDF membranes. Membranes were probed by incubation with appropriate antibodies. These included mouse anti-myc (9E10, Gentaur) and goat anti-mouse Fc HRP (Jackson ImmunoResearch) for the detection of VHHs with and without scFc tags; biotinylated-“O” (produced in-house), rabbit anti-beta-catenin (6B3, Cell Signalling Technologies) and rabbit anti-penta HIS (Bethyl laboratories) followed by streptavidin HRP (Jackson ImmunoResearch) or anti-rabbit Fc HRP (Jackson ImmunoResearch) respectively for the detection of beta-catenin. All membranes were developed with Pico Super Signal ECL (Pierce) for 5 minutes, and images captured using ImageQuant LAS 4000 (GE Healthcare) and densitometry of resulting bands analyzed by ImageQuant TL analysis software.

Beta-catenin fragment generation: BL21 Star™ DE3 cells (Invitrogen) expressing each of the beta-catenin constructs were grown in antibiotic selective 2×TY media supplemented with IPTG (final concentration of 0.3 mM), shaking overnight. Cells from the overnight culture were harvested by centrifugation at 4,000×g for 5 minutes. The pelleted cells were lysed with 1×Bugbuster® (Novagen) containing Lysonase (Novagen) in 20 mM sodium phosphate, 500 mM NaCl, 20 mM imidazole, 2 mM DTT) by rolling at room temperature for 20 minutes.

SPR-Affinity measurements: Affinity measurements were performed using a Biacore™T100. Streptavidin was immobilised onto the carboxymethylated dextran coated gold surface of a CM5 sensor chip using amine coupling chemistry, as per manufacture's instructions. The concentrations of streptavidin used in flow cells 1˜4 respectively were 0 μg/mL, 5 μg/mL, 15 μg/mL, 45 μg/mL in 10 mM NaOAc pH5. Biotinylated beta-catenin was then captured on flow cells 2-4 whilst flow cell 1 was the control reference cell. Samples of VHH-scFc at 1 in 2, 1 in 5, 1 in 10, 1 in 20 and 1 in 40 dilutions in HBS-EP were passed over all flow cells for 5 minutes at 30 μL/minute. Buffer blanks were run between every sample set and control transfection supernatant samples were included in each run. Between each sample the chip was regenerated by the addition of 30 μL of 2M guanidine hydrochloride followed by 30 μL of 40 mM HCl. Affinity constants were determined by the fitting of resulting data using BiaEvalution software with a Langmuir 1:1 association method, associations were rejected if the Chi2 value was greater than 1; if the residual data points were not randomly distributed or if the residuals were greater than +/−2 RU. The association data were plotted for the most dilute samples which still gave a RU capture response of at least 40RU. A minimum of four different combinations of dilutions and different beta-catenin coated flow cells were used to calculate association ‘on-rate’ (ka) or dissociation data ‘off-rate’ (kd), and used to calculate KD=kd/ka.

### Example 1—Production of a VHH Anti-Beta-Catenin Immune Phage Display Library

Two Llamas were immunised as described above. After dose five, as sera titres did not further improve, immunisation was stopped. Although the serum response ELISA suggested the production of beta-catenin binding antibodies, it did not provide information on the isotypes of antibodies produced, because the anti-llama IgG antibody recognised both conventional and HCAb isotypes. Antibody isotypes with specificity for beta-catenin were identified by immuno-precipitation with beta-catenin coated beads and compared to the amount and isotypes of antibody precipitated with uncoated beads as a control, using standard protocols. The results suggested that enrichment had occurred and greater amounts of conventional and heavy chain only antibodies were captured when beta-catenin was present. The llama sera immuno-precipitation experiment indicated that both llamas had made an immune response to beta-catenin, which included HCAbs.

A llama immune phage library was then produced (as described in the material and method section above). Following on from the monoclonal phage rescue, a large scale polyclonal phage rescue was completed of the whole library and the sequences were further categorised into subfamilies based on the criteria of Harmsen (Harmsen et al., 2000) (see Table 2 below). Both conventional IgGs and HCAbs capable of binding beta-catenin were identified in post immunisation sera and members of each of the VHH subfamilies 1-3 previously found in llamas were identified.

TABLE 2
Subfamily classification of llama VHH sequences recorded
from sequencing of the anti beta-catenin immune phage
library. The percentages are based on 124 analysed sequences,
according to the classification scheme.

| Sub-Family Classification | Percentage of Library |
|---|---|
| VHH1 | 41 |
| VHH2 | 15 |
| VHH3 | 19 |
| VHH4 | 0 |
| Conventional VH | 8 |
| Unclassified | 17 |

### Example 2—Identification of Intrabody Variable Regions

Selection of antigen specific antibodies from a phage library typically involves iterative rounds of a process commonly known as panning. Multiple rounds of the panning process are usually required due to inefficient removal of all non-specific antigen binding phage particles during panning.

The aim of this work was not to identify any anti-beta-catenin antibodies, but to select a diverse array of specific anti-beta-catenin VHHs which bound to intracellular beta-catenin in its native cellular conformation. However, such selection would have been technically challenging using conventional phage display. Therefore, due to practical considerations recombinant beta-catenin was used both displayed on immunotubes and in-solution to maximise the number of available epitopes. Each of these panning conditions had also been extended to better mimic the intracellular environment for instance by the addition of reducing agent.

To identify individual antibody variable regions with specificity for beta-catenin, a total of 855 small scale phage rescues and antibody-pIII inductions were performed for individual colonies in 96 well block format. These methods screened for binding from samples in the rescued antibody-phage fusion format and the induced antibody-pIII format. In addition, each sample in both formats was tested for binding to beta-catenin captured on the ELISA plate as well as streptavidin coated plates, to check if the signal recorded was caused by beta-catenin binding. The phage rescue samples were also analysed for phage-VHH production. From this work, 73 beta-catenin-binding promising sequences were identified. After cloning into a mammalian expression vector, antibodies were expressed intracellularly and tested for their ability to modify the function of beta-catenin. A total of 61 out of the 73 sequences were successfully cloned.

In order to assess anti-beta-catenin neutralizing activity of these antibodies, Wnt signalling activity assays were performed using HEK293 cells previously stably transfected with a reporter construct, which contained 16×TCF/LEF binding sites upstream of the firefly luciferase reporter gene. Intrabodies were expressed intracellularly by transfection of vector DNA, and any inhibition of beta-catenin co-transcriptional activity was detected by a decrease in the amount of luciferase produced. This assay allowed direct identification of intrabodies that modulate canonical Wnt signalling.

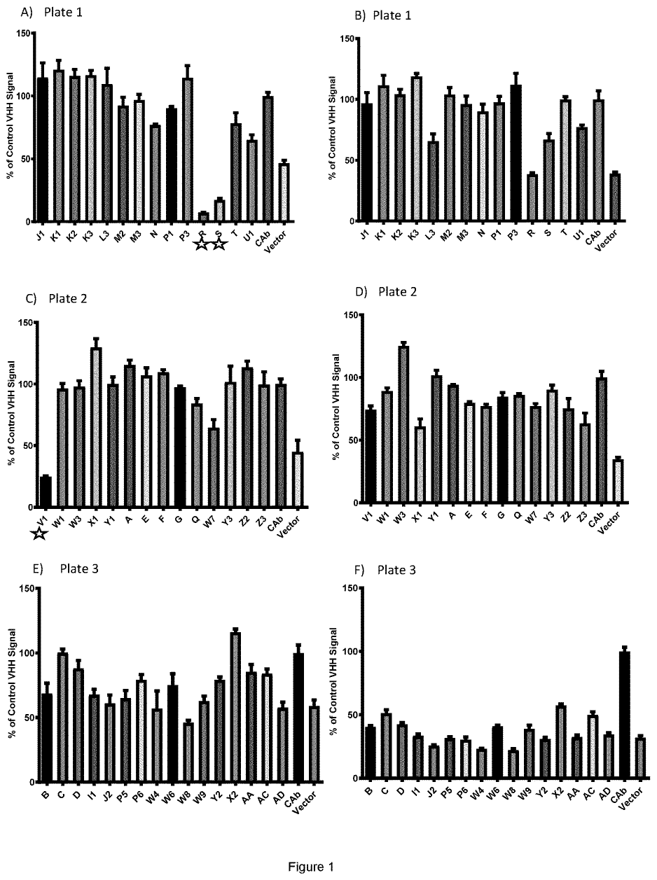

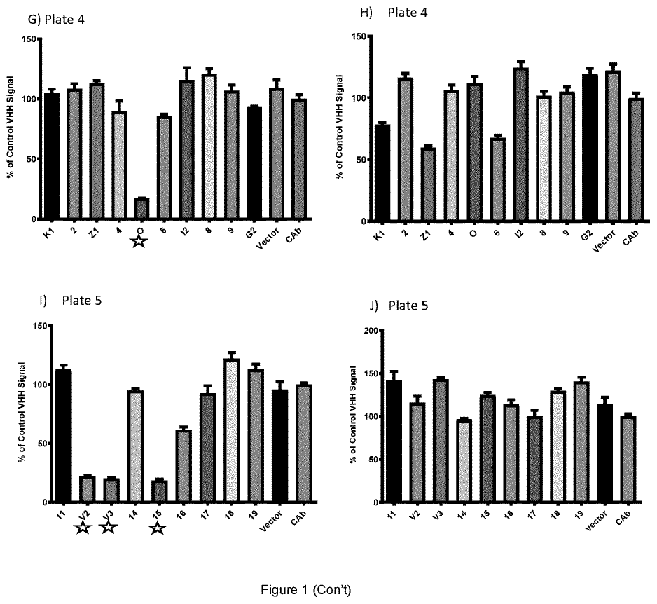

The sub-cloned VHH intrabodies were transfected in triplicate at 100 ng/well, into HEK293 bioassay cells. Firefly luciferase expression was stimulated by co-transfection with 50 ng of LGR6 PPSP motif A peptide (Tamai et al. 2004) encoding DNA/well. After 40 hours incubation at 37° C. the cells were lysed, and firefly and Renilla luciferase activity were measured independently. All plates were transfected in duplicate so that one plate could also be used to visualise the levels of VHH expressed by Western blotting. Sixty-one VHH intrabodies were analysed in this way, the results of these are shown in FIG. 1.

Intrabodies were screened for intracellular function against beta-catenin co-transcriptional activity in the HEK293 bioassay. All luciferase activity signals were plotted as a percentage of the control VHH (CAb signal) which was transfected on each screening plate, to facilitate the comparison of samples between plates. Some sequences clearly affected the individual luciferase activity signals, for instance intrabodies 6, Z1-3 and R all produced a marked decrease in Renilla luciferase activity, indicating cellular toxicity. Intrabodies X1 and X2 both showed some stimulation of firefly luciferase activity over the CAb signal, and intrabodies V1-3 all showed strong inhibition of the firefly luciferase signal. Also, the most diverse family based on sequence was the largest intrabody family W, which also produced the most diverse luciferase activity signals based on bioassay results. Out of the 61 tested intrabodies, seven VHHs were selected as intrabodies for further analysis (the whole V intrabody family V1, V2 and V3, and the intrabodies R, S, O and 15). These intrabodies were selected because they all showed very strong inhibition of the firefly luciferase activity signal, ≤25% of the stimulated luciferase signal recorded by control VHH (see FIGS. 1A, C, E, G and I). Intrabodies V2, V3, O and 15 all also maintained a similar Renilla luciferase activity signal as that recorded by CAb, showing that the inhibition of firefly luciferase activity was specific (see FIGS. 1B, D, F, H and J). Intrabodies V1 and S did show some inhibition of Renilla luciferase activity, however it was not as strong as the firefly luciferase activity inhibition, and it was never below the signal seen for non-VHH containing mammalian expression vector control Renilla luciferase activity included on each plate. Intrabody R did show greater than 50% inhibition of the Renilla luciferase signal signifying that its inhibitory activity on firefly luciferase activity may not be specific. All seven intrabodies were moved onto the next stage of screening.

These intrabodies (selected from the primary screen and shown to express by Western blotting; data not shown), were then screened again in the bioassay using a different stimulus, WNT1 transfected DNA. Two beta-catenin-binding intrabodies (W3 and C), which had previously not demonstrated inhibition in the bioassay but which did bind beta-catenin, were included as ‘negative’ controls on each plate, and inhibitory intrabodies R, S and V1 were included as ‘positive’ controls on each plate. In addition, a variety of other intrabodies, which had previously not affected the bioassay, were included over the two screening plates to test if VHHs generally behaved in the same manner with both bioassay stimulations.

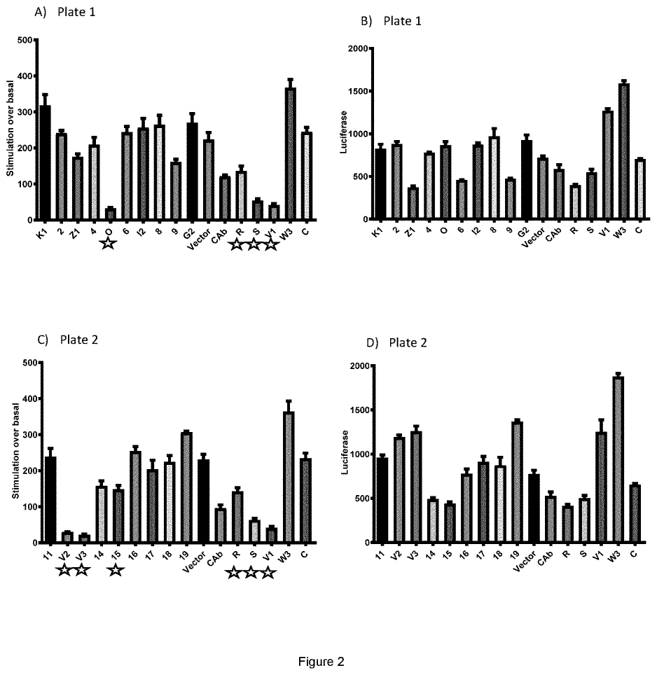

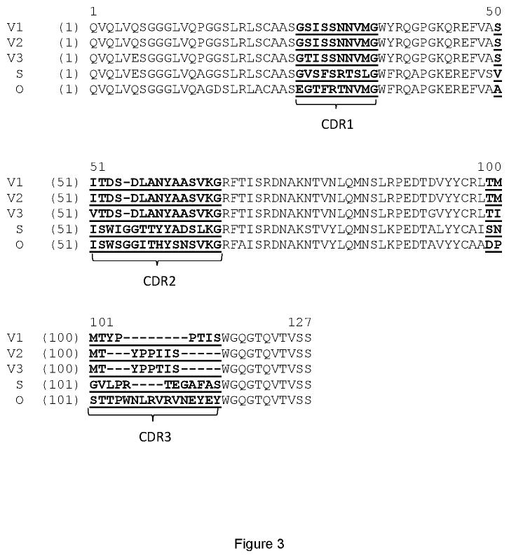

The results of the bioassay run with WNT1-stimulation (FIG. 2) showed that again intrabodies V1, V2, V3, S and O (alternatively herein named IBV1, IBV2, IBV3, IBS and IBO) demonstrated inhibition of firefly luciferase activity compared to all the other intrabodies transfected on their respective plates. IBV1, IBV2, IBV3 and IBO also showed no inhibition of Renilla luciferase activity. Intrabody S again showed some inhibition of Renilla luciferase activity but not to the same level as firefly luciferase activity inhibition. Intrabodies R and 15 did not appear capable of inhibiting the WNT1-induced firefly luciferase activity more than they did the constitutively expressed Renilla luciferase activity. This implied that their activity was not specific to the inhibition of beta-catenin-mediated firefly luciferase production, and they were therefore not investigated further. The sequences of the variable regions of the five intrabodies capable of inhibiting beta-catenin-mediated firefly luciferase production are shown in FIG. 3. These intrabodies belong to three families, based on CDR3 variability.

### Example 3—Characterisation of Function Modifying Intrabodies

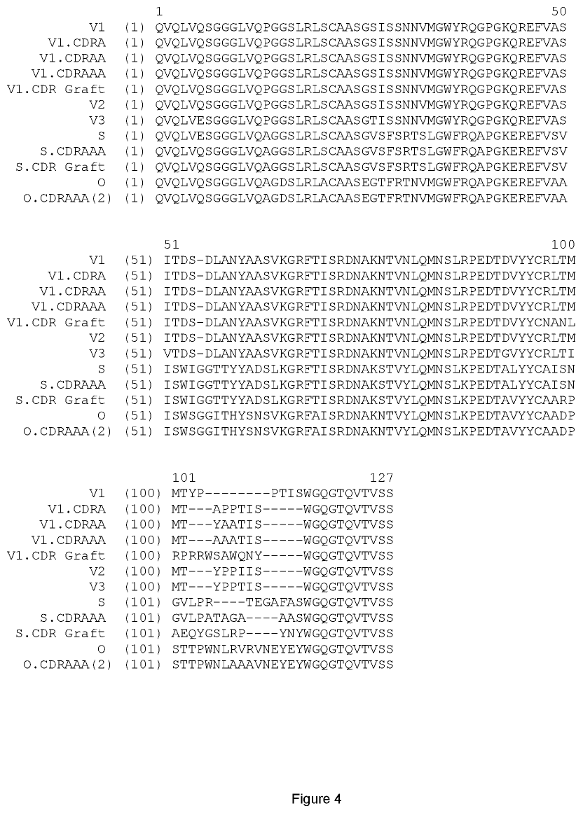

To confirm that intrabody activity was dependent on antigen binding, the five more promising antibodies identified in Example 2 (parents) were mutated in their CDR3, in an attempt to perturb binding to beta-catenin with minimal disruption to the rest of the VHH domain. These mutations were selected by careful study of CDR3 for prime binding residues such as tyrosines based on literature. In addition, for each of the parent, complete CDR3 grafts were also engineered. The particular CDR3 to graft was identified by aligning all the available sequences, previously identified, against the parent intrabody sequences in turn, and a CDR3 of similar length was selected from the most homologous antibodies (FIG. 4).

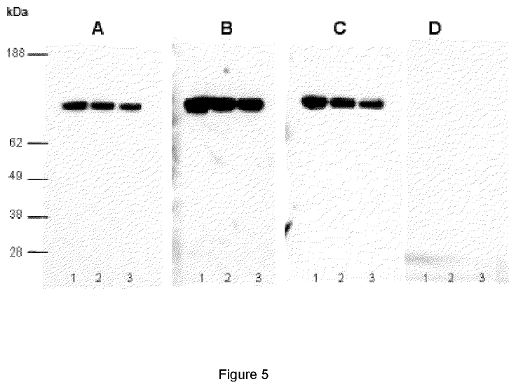

Reduced beta-catenin samples were run on four 4-12% bis-tris gels and transferred to PVDF membranes. The membranes were probed with IBV1, IBS, IBO and IBV1.CDRAAA as a control, all in the VHH-scFc format, and revealed with goat anti-mouse HRP (FIG. 5). The presence of beta-catenin was successfully detected by all three parent VHHs and not by the control mutant IBV1.CDRAAA. These data strongly suggested that all three parent VHHs bound to linear rather than conformational epitopes on beta-catenin.

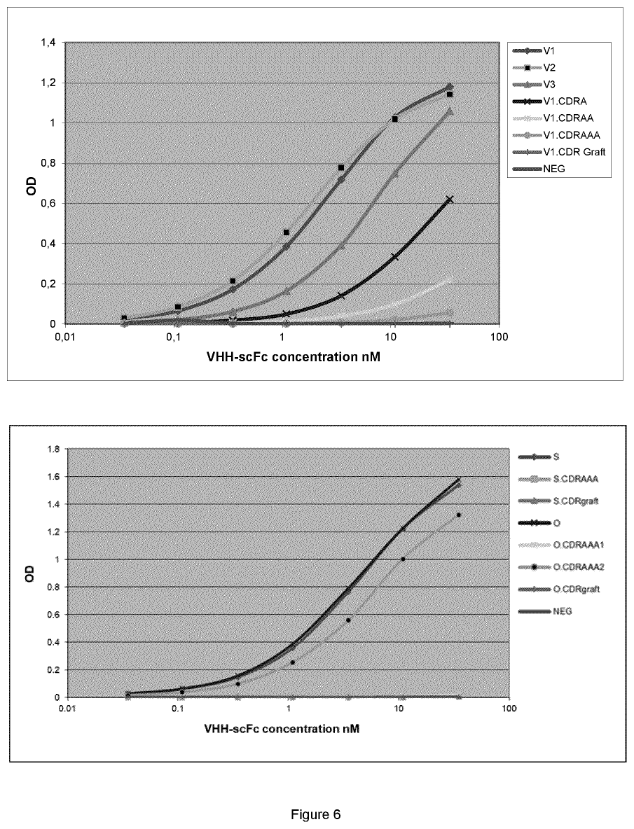

The binding of the parent intrabodies and CDR3 mutants to recombinant β-catenin was also tested by ELISA. All of them showed binding to β-catenin (FIG. 6). Of the various CDR3 mutants only V1.CDRA, V1.CDRAA and O.CDRAAA (2) showed significant binding to β-catenin. O.CDRAAA (1) demonstrated all the desired characteristics of such a control VHH for the parent IBO. IBV1.CDRAAA and IBS.CDRAAA also appeared to be suitable CDR3 mutant controls for their respective parent except for their lower expression levels within the bioassay. This is not uncommon in conventional antibody expression and single point mutations in antibody CDRs leading to over a 100-fold reduction in expression have been previously reported.

Neither intrabody O nor intrabody O.CDRAAA (1) had an inhibitory effect on Renilla luciferase activity in the bioassay, implying that the expression of these VHHs was not detrimental to cell viability. The decrease in Renilla luciferase production for IBV1.CDRAAA and IBS.CDRAAA compared to IBV1 and IBS indicated that the intracellular expression of these CDR3 mutants may well have reduced cell viability. This again demonstrated the differential effects that expression of individual VHH sequences can have on mammalian cells.

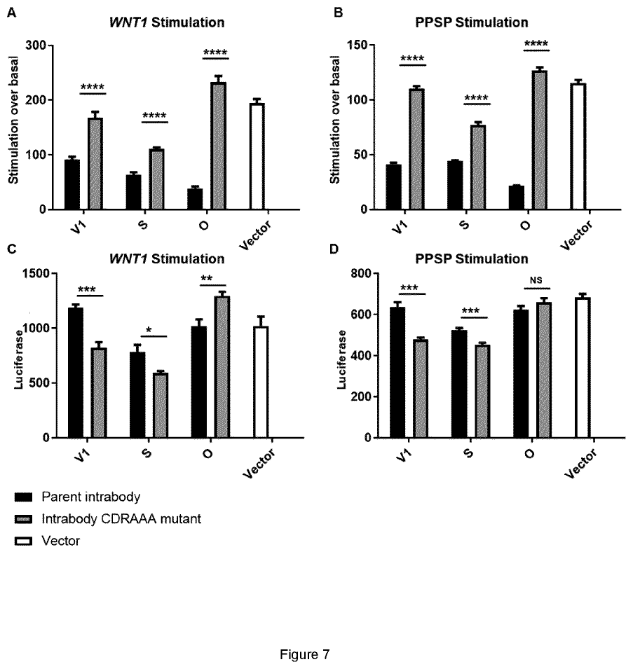

Upon repeated testing, parent intrabodies V, S and O showed consistent statistically significant inhibition of both PPSP- and Wnt1-induced signalling when compared to their selected CDR3 mutant control intrabodies. The inhibition of luciferase activity by the parent intrabodies was specific to beta-catenin-induced firefly luciferase production, and not to Renilla luciferase activity (see FIG. 7). Although some statistical significance was recorded for the difference in Renilla luciferase activity between parent intrabodies and their respective CDR3 mutant control intrabodies, this was generally not inhibition by the parent intrabodies but rather by the CDR3 mutant control intrabodies, i.e. parent intrabodies had greater Renilla luciferase activity than the CDR3 mutant control intrabodies. When the data were re-plotted to take the relative changes in Renilla luciferase activity into account the percentage inhibition of intrabodies V1 and S rose by an average of 10%. These data confirmed that the inhibition of firefly luciferase production by intrabodies V, S and O was a result of their ability to modulate beta-catenin's function, and not a result of off-target effects caused by the expression of the VHH domain inside the cell. This demonstrated that function-modifying intrabodies to the Wnt signalling pathway had been successfully produced.

It was interesting to note that some of the engineered CDR3 mutants retained specific binding to beta-catenin. Mutants IBV1.CDRA and IBO.CDRAAA (2) retained binding to beta-catenin as detected by ELISA and SPR. They also demonstrated specific inhibition of firefly luciferase activity in the bioassay. IBO.CDRAAA (2) retained much of the bioassay activity of IBO despite the triple Ala substitutions in CDR3. The Ala substitutions included a pair of bulky, charged arginine residues which could have altered the structure and binding capacity of CDR3. Clearly this was not the case (as supported by the literature) which implied that arginines in CDR3 do not generally contribute to affinity for antigen or necessarily correlate with the specificity of antibody-antigen interactions. It appeared that the off-rate data for VHH binding to beta-catenin correlated well with the activity of VHHs in the bioassay; for instance IBV1.CDRA and IBO.CDRAAA (2) had the best off rates of all the mutants and the most activity in the bioassay (data not shown). This was most likely a result of the high concentration of cellular beta-catenin or intrabody, which meant that regardless of on-rate, the intrabodies bound beta-catenin, and the determining factor for activity was how long they remained bound. Slow on-rate could also be the reason why more binding was recorded in the ELISA than the by SPR for some CDR3 mutants, for instance IBV1.CDRAA. The affinity values for VHH families V, S and O were determined by SPR and by ELISA. KD values as determined by SPR can be found in Table 3 below.

TABLE 3
SPR determined ka and kd values for parent VHHs. Values determined
by fitting of multiple SPR traces using BIAevalution software

| Sample | Ka (M − 1s − 1) | Kd (s − 1) | KD (nM) |
|---|---|---|---|
| V1 | 25100 | 0.000163 | 6.49 |
| V2 | 36900 | 0.000165 | 4.47 |
| V3 | 38500 | 0.000169 | 4.38 |
| S | 65600 | 0.000248 | 3.78 |
| O | 71100 | 0.000260 | 3.65 |

All the KD values, obtained by Elisa, highlighted affinity values below 10 nM. The affinity values obtained by SPR recorded showed that the best affinity were obtained for S and O. For the intrabodies from family V, SPR demonstrated a slightly better affinity for V2 and V3 compared to V1.It would appear that the single amino acid changes between IBV1, IBV2 and IBV3 did have a slight effect on their relative affinities. Both IBV2 and IBV3 contained an additional isoleucine in CDR3 rather than a threonine or methionine respectively, and both have even slightly greater affinity for beta-catenin than IBV1.

The SPR studies showed that IBV1, IBV2, IBV3, IBO and IBS all ‘cross blocked’ each other, i.e. it was not possible to bind one intrabody to beta-catenin if another was already bound.

In summary three intrabody families have been discovered which specifically affect beta-catenin co-transcriptional activity by virtue of CDR3-mediated interactions. It appeared that each of the parent VHHs ‘cross blocked’ each other. The most suitable intrabody and CDR mutant intrabody control pair was intrabodies IBO and IBO.CDRAAA (1), this pair also demonstrated the greatest percentage inhibition (83%) of firefly luciferase production in the bioassay. Hence intrabodies IBO and IBO.CDRAAA (1) were taken forward for further studies.

### Example 4—Mode of Action

As shown in the previous examples, the anti-beta-catenin VHH of the invention have consistently shown inhibition of the Wnt signalling pathway as measured by the inhibition of, Wnt1 or PPSP-stimulated, firefly luciferase production in the HEK293 bioassay.

To better understand the mode of action of the anti-beta-catenin herein identified, one of them, (antibody IBO) has been further studied. Two further stimuli, Wnt3a-conditioned media and LiCl2, as well as the use of an alternative bioassay have been investigated. Wnt3a and Wnt1 are both thought to signal in a similar canonical fashion but can sometimes use different sets of Frizzled co-receptors. LiCl2 is a potent inhibitor of GSK3β, preventing it from phosphorylating beta-catenin. IBO showed a 66% inhibition of beta-catenin co-transcriptional activity in the HEK293 bioassay stimulated with Wnt3a-conditioned media, and 65% inhibition when stimulated with LiCl2 compared to the CDR3 control mutant intrabody IBO.CDRAAA (1). The fact that IBO could still inhibit the firefly luciferase signal signifies that the intrabody IBO-beta-catenin interaction is downstream of the GSK3β-beta-catenin interaction. Using a series of truncated beta-catenin fragments and Western blotting, IBO was shown to bind between residues 138 and 390 on beta-catenin. This region coincides with the binding regions of Pontin52, Bcl-9 and TCF to beta-catenin. Importantly IBO was shown to bind endogenous beta-catenin, and the binding site was demonstrated to reside between residues 138 and 390. Initial indications were that intrabody IBO specifically disrupts beta-catenin co-transcriptional activity without affecting its role at the plasma membrane (data not shown).

### OVERALL CONCLUSION

In summary, this present invention describes novel VHH-based antibodies and active fragments thereof that are able to disrupt the biological activity of b-catenin inside the cell. These VHH-based antibodies and active fragments thereof can therefore be used as intrabodies for the treatment of disorders involving beta-catenin or beta-catenin pathway, such as those described in the present description as a whole.

### REFERENCES

1) Biocca et al., 1995, Biotechnology, 13:1110-1115
2) Stocks et al., 2005, Curr. Opin. Chem. Biol., 9:359-365
3) Serruys et al., 2009, Hepatology, 49:39-49
4) Vercruysse et al., 2010, J. Biol. Chem., 285:21768-21780
5) Ben-Ze'ev and Geiger, 1998, Curr. Opin. Cell Biol., 10:629-639
6) Morin et al., 1999, BioEssays, 21:1021-1030
7) Newnham et al., 2015, mAbs, 7(1):180-191;
8) Miyaoka et al., 1999, Schizophr. Res., 38:1-6.
9) Grant et al., 2006, Nat. Genet., 38:320-323
10) Sen et al., 2000, PNAS, 97:2791-2796
11) Lammi et al., 2004, Am. J. Hum. Genet., 74:1043-1050
12) Gong et al., 2001, Cell, 107:513-523
13) Toomes et al., 2004, Am. J. Hum. Genet., 74:721-730.
14) Bienz and Clevers, 2000, Cell, 103:311-320
15) McCrea et al., 1993, J. Cell Biol., 123:477-484
16) WO2021015419
17) Kabat et al., 1991, Sequences of Proteins of Immunological Interest, NIH publication No. 91-3242
18) Chothia, C. and Lesk, A. M., 1987, J. Mol. Biol., 196:901-917
19) Angal et al., 1993, Mol Immunol, 30:105-108
20) Kashmiri et al., 2005, Methods, 36, 25-34
21) van den Berg and Dowdy, 2011, Curr. Opin. Biotechnol., 22:888-893
22) Lafaye et al., 2011 (US2011250211)
23) Yildiz et al., 2011, Curr. Opin. Biotechnol., 22:901-908
24) Shi Kam et al., 2004, J. Am. Chem. Soc., 126:6850-6851
25) Xing Y et al., 2008, Structure, 16:478-487
26) Harmsen et al., 2000, Mol. Immunol., 37:579-590
27) Tamai et al., 2004, Mol. Cell, 13:149-156

1. An antibody that specifically binds to beta-catenin, wherein the antibody comprises a heavy chain variable region comprising:
i. a CDR-H1 of SEQ ID NO: 1, 35 or 36; a CDR-H2 of SEQ ID NO: 2, 37 or 38; and a CDR-H3 of SEQ ID NO: 3, 39, 40, 41, 42, 43, 44 or 45;
ii. a CDR-H1 of SEQ ID NO: 4; a CDR-H2 of SEQ ID NO: 5 and a CDR-H3 of SEQ ID NO: 6, 44, 45 or 46;
iii. a CDR-H1 of SEQ ID NO: 7; a CDR-H2 of SEQ ID NO: 8 and a CDR-H3 of SEQ ID NO: 9, 47, 48, 49 or 50; or
iv. CDR-H1, CDR-H2 and CDR-H3 sequences having at least 70%, 80%, 90%, 91%, 92%, 93%, 94%, 95%, 96%, 97%, 98%, or 99% identity or similarity to any of the sequences listed in any one of i. to iii.

2. The antibody according to claim 1, wherein said antibody further comprises framework regions (FRs) comprising or consisting of:
i. A FR1 comprising SEQ ID NO: 10, 15, or 18 or a sequence having at least 70%, 80%, 90%, 91%, 92%, 93%, 94%, 95%, 96%, 97%, 98%, or 99% identity thereof;
ii. A FR2 comprising SEQ ID NO: 11, 16 or 19 or a sequence having at least 70%, 80%, 90%, 91%, 92%, 93%, 94%, 95%, 96%, 97%, 98%, or 99% identity thereof;
iii. A FR3 comprising SEQ ID NO: 12, 13, 17 or 20 or a sequence having at least 70%, 80%, 90%, 91%, 92%, 93%, 94%, 95%, 96%, 97%, 98%, or 99% identity thereof; and
iv. A FR4 comprising SEQ ID NO: 14 or a sequence having at least 70%, 80%, 90%, 91%, 92%, 93%, 94%, 95%, 96%, 97%, 98%, or 99% identity thereof.

3. The antibody according to any one of the preceding claims, wherein the antibody has a heavy chain variable region comprising any one of SEQ ID NO: 21, 22, 23, 24, 25, 28, 31 or 33 or a sequence having at least 70%, 80%, 90%, 91%, 92%, 93%, 94%, 95%, 96%, 97%, 98%, or 99% identity thereof.

4. The antibody according to any one of the preceding claims, wherein the antibody is a VHH or an Hcab.

5. The antibody according to any one of the preceding claims wherein the antibody is an intrabody.

6. The antibody according to any one of the preceding claims wherein the antibody is chimeric or humanized.

7. The antibody according to any one of the preceding claims, wherein the antibody has a heavy chain comprising any one of SEQ ID NO: 59, 60, 61 or 62 or a sequence having at least 70%, 80%, 90%, 91%, 92%, 93%, 94%, 95%, 96%, 97%, 98%, or 99% identity thereof.

8. The antibody according to any one of the preceding claims, wherein said antibody i) blocks beta-catenin binding to any one or more of Pontin52, Bcl-9 and lymphoid-enhancing factor-1 (LEF-1)/T-cell factor (TCF) family (TCF) and/or ii) neutralises or inhibits beta-catenin mediated pathway.

9. The antibody according to any one of the preceding claims, wherein said antibody has an equilibrium dissociation constant (KD) of less than 20 nM for beta-catenin.

10. The antibody according to any one of the preceding claims which specifically binds to human beta-catenin on an epitope located between residues 138-390 of SEQ ID NO.51.

11. An antibody that cross-competes with the antibody of any one of the preceding claims for binding to beta-catenin.

12. An isolated polynucleotide encoding the antibody according to any one of claims 1 to 11.

13. A cloning or expression vector comprising the polynucleotide according to claim 12.

14. A host cell comprising the polynucleotide according to claim 12 or the expression vector according to claim 13.

15. A process for the production of an antibody according to any one of claims 1 to 11, comprising culturing the host cell according to claim 14 under suitable conditions for producing the antibody and isolating the antibody.

16. A pharmaceutical composition comprising the antibody according to any one of claims 1 to 11, or a polynucleotide according to claim 12, and one or more pharmaceutically acceptable carriers, excipients and/or diluents.

17. The antibody according to any one of claims 1 to 11, the polynucleotide according to claim 12 or the pharmaceutical composition according to claim 16 for use in therapy.

18. The antibody according to any one of claims 1 to 11, the polynucleotide according to claim 12 or the pharmaceutical composition according to claim 16 for use in the treatment of a disease or a condition characterized by over expression of beta-catenin.

19. The antibody according to any one of claims 1 to 11 and 17-18, the polynucleotide according to claim 12, 17 or 18 or the pharmaceutical composition according to claims 16 to 18 for use in the treatment of (i) a cancer such as (colorectal cancers, adenomatous polyposis (FAP), colon cancer, melanoma, hepatocellular carcinoma, ovarian cancer, endometrial cancer, medulloblastoma pilomatricomas, and prostate cancer, (ii) a neurodegenerative disorder, such as Huntington's disease, Alzheimer's disease, Parkinson's disease, prion diseases, multiple sclerosis, amyotrophic lateral sclerosis or schizophrenia, or (iii) any one of bone density disorders, schizophrenia, type II diabetes, rheumatoid arthritis, oligodentonia, osteoporosis-pseudoglioma syndrome; familial exudative vitreoretinopathy or yet vascular calcification.
